SEQUENCE LISTING (46 sequences)

SEQ ID NO: 1 - DNA, 1607 bp, Ehrlichia canis
  nucleic acid sequence of E. canis p28-7
attttatttattaccaatcttatataatatattaaatttctcttacaaaaatctctaatgttttatacctaatatatatattctggcttgtatctactttgcacttccactattgttaatttattttcactattttaggtgtaatatgaattgcaaaaaaattcttataacaactgcattaatatcattaatgtactctattccaagcatatctttttctgatactatacaagatggtaacatgggtggtaacttctatattagtggaaagtatgtaccaagtgtctcacattttggtagcttctcagctaaagaagaaagcaaatcaactgttggagtttttggattaaaacatgattgggatggaagtccaatacttaagaataaacacgctgactttactgttccaaactattcgttcagatacgagaacaatccatttctagggtttgcaggagctatcggttactcaatgggtggcccaagaatagaattcgaaatatcttatgaagcattcgacgtaaaaagtcctaatatcaattatcaaaatgacgcgcacaggtactgcgctctatctcatcacacatcggcagccatggaagctgataaatttgtcttcttaaaaaacgaagggttaattgacatatcacttgcaataaatgcatgttatgatataataaatgacaaagtacctgtttctccttatatatgcgcaggtattggtactgatttgatttctatgtttgaagctacaagtcctaaaatttcctaccaaggaaaactgggcattagttactctattaatccggaaacctctgttttcatcggtgggcatttccacaggatcataggtaatgagtttagagatattcctgcaatagtacctagtaactcaactacaataagtggaccacaatttgcaacagtaacactaaatgtgtgtcactttggtttagaacttggaggaagatttaacttctaattttattgttgccacatattaaaaatgatctaaacttgtttttawtattgctacatacaaaaaaagaaaaatagtggcaaaagaatgtagcaataagaggggggggggggaccaaatttatcttctatgcttcccaagttttttcycgctatttatgacttaaacaacagaaggtaatatcctcacggaaaacttatcttcaaatattttatttattaccaatcttatataatatattaaatttctcttacaaaaatcactagtattttataccaaaatatatattctgacttgcttttcttctgcacttctactatttttaatttatttgtcactattaggttataataawatgaattgcmaaagatttttcatagcaagtgcattgatatcactaatgtctttcttacctagcgtatctttttctgaatcaatacatgaagataatataaatggtaacttttacattagtgcaaagtatatgccaagtgcctcacactttggcgtattttcagttaaagaagagaaaaacacaacaactggagttttcggattaaaacaagattgggacggagcaacactaaaggatgcaagcwgcagccacacawtagacccaagtacaatg

SEQ ID NO: 2 - protein, 278 aa, Ehrlichia canis
  amino acid sequence of E. canis p28-7 protein
MNCKKILITTALISLMYSIPSISFSDTIQDGNMGGNFYISGKYVPSVSHFGSFSAKEESKSTVGVFGLKHDWDGSPILKNKHADFTVPNYSFRYENNPFLGFAGAIGYSMGGPRIEFEISYEAFDVKSPNINYQNDAHRYCALSHHTSAAMEADKFVFLKNEGLIDISLAINACYDIINDKVPVSPYICAGIGTDLISMFEATSPKISYQGKLGISYSINPETSVFIGGHFHRIIGNEFRDIPAIVPSNSTTISGPQFATVTLNVCHFGLELGGRFNF

SEQ ID NO: 3 - DNA, 849 bp, Ehrlichia canis
  mat_peptide: nucleic acid sequence of p28-5
atgaattgtaaaaaagttttcacaataagtgcattgatatcatccatatacttcctacctaatgtctcatactctaacccagtatatggtaacagtatgtatggtaatttttacatatcaggaaagtacatgccaagtgttcctcattttggaattttttcagctgaagaagagaaaaaaaagacaactgtagtatatggcttaaaagaaaactgggcaggagatgcaatatctagtcaaagtccagatgataattttaccattcgaaattactcattcaagtatgcaagcaacaagtttttagggtttgcagtagctattggttactcgataggcagtccaagaatagaagttgagatgtcttatgaagcatttgatgtgaaaaatccaggtgataattacaaaaacggtgcttacaggtattgtgctttatctcatcaagatgatgcggatgatgacatgactagtgcaactgacaaatttgtatatttaattaatgaaggattacttaacatatcatttatgacaaacatatgttatgaaacagcaagcaaaaatatacctctctctccttacatatgtgcaggtattggtactgatttaattcacatgtttgaaactacacatcctaaaatttcttatcaaggaaagctagggttggcctacttcgtaagtgcagagtcttcggtttcttttggtatatattttcataaaattataaataataagtttaaaaatgttccagccatggtacctattaactcagacgagatagtaggaccacagtttgcaacagtaacattaaatgtatgctactttggattagaacttggatgtaggttcaacttc

SEQ ID NO: 4 - protein, 283 aa, Ehrlichia canis
  amino acid sequence of p28-5 protein
MNCKKVFTISALISSIYFLPNVSYSNPVYGNSMYGNFYISGKYMPSVPHFGIFSAEEEKKKTTVVYGLKENWAGDAISSQSPDDNFTIRNYSFKYASNKFLGFAVAIGYSIGSPRIEVEMSYEAFDVKNPGDNYKNGAYRYCALSHQDDADDDMTSATDKFVYLINEGLLNISFMTNICYETASKNIPLSPYICAGIGTDLIHMFETTHPKISYQGKLGLAYFVSAESSVSFGIYFHKIINNKFKNVPAMVPINSDEIVGPQFATVTLNVCYFGLELGCRFNF

SEQ ID NO: 5 - DNA, 840 bp, Ehrlichia canis
  mat_peptide: nucleic acid sequence of p28-6
atgaattgcaaaaaaattcttataacaactgcattaatgtcattaatgtactatgctccaagcatatctttttctgatactatacaagacgataacactggtagcttctacatcagtggaaaatatgtaccaagtgtttcacattttggtgttttctcagctaaagaagaaagaaactcaactgttggagtttttggattaaaacatgattggaatggaggtacaatatctaactcttctccagaaaatatattcacagttcaaaattattcgtttaaatacgaaaacaacccattcttagggtttgcaggagctattggttattcaatgggtggcccaagaatagaacttgaagttctgtacgagacattcgatgtgaaaaatcagaacaataattataagaacggcgcacacagatactgtgctttatctcatcatagttcagcaacaagcatgtcctccgcaagtaacaaatttgttttcttaaaaaatgaagggttaattgacttatcatttatgataaatgcatgctatgacataataattgaaggaatgcctttttcaccttatatttgtgcaggtgttggtactgatgttgtttccatgtttgaagctataaatcctaaaatttcttaccaaggaaaactaggattaggttatagtataagttcagaagcctctgtttttatcggtggacactttcacagagtcataggtaatgaatttagagacatccctgctatggttcctagtggatcaaatcttccagaaaaccaatttgcaatagtaacactaaatgtgtgtcactttggcatagaacttggaggaagatttaacttc

SEQ ID NO: 6 - protein, 280 aa, Ehrlichia canis
  amino acid sequence of p28-6 protein
MNCKKILITTALMSLMYYAPSISFSDTIQDDNTGSFYISGKYVPSVSHFGVFSAKEERNSTVGVFGLKHDWNGGTISNSSPENIFTVQNYSFKYENNPFLGFAGAIGYSMGGPRIELEVLYETFDVKNQNNNYKNGAHRYCALSHHSSATSMSSASNKFVFLKNEGLIDLSFMINACYDIIIEGMPFSPYICAGVGTDVVSMFEAINPKISYQGKLGLGYSISSEASVFIGGHFHRVIGNEFRDIPAMVPSGSNLPENQFAIVTLNVCHFGIELGGRFNF

SEQ ID NO: 7 - protein, 133 aa, Ehrlichia canis
  partial amino acid sequence of p28-5 protein
MNCKKVFTISALISSIYFLPNVSYSNPVYGNSMYGNFYISGKYMPSVPHFGIFSAEEEKKKTTVVYGLKENWAGDAISSQSPDDNFTIRNYSFKYASNKFLGFAVAIGYSIGSPRIEVEMSYEAFDVKNQGNN

SEQ ID NO: 8 - protein, 287 aa, Ehrlichia canis
  amino acid sequence of p28-4 protien
MKYKKTFTVTALVLLTSFTHFIPFYSPARASTIHNFYISGKYMPTASHFGIFSAKEEQSFTKVLVGLDQRLSHNIINNNDTAKSLKVQNYSFKYKNNPFLGFAGAIGYSIGNSRIELEVSHEIFDTKNPGNNYLNDSHKYCALSHGSHICSDGNSGDWYTAKTDKFVLLKNEGLLDVSFMLNACYDITTEKMPFSPYICAGIGTDLISMFETTQNKISYQGKLGLNYTINSRVSVFAGGHFHKVIGNEFKGIPTLLPDGSNIKVQQSATVTLDVCHFGLEIGSRFFF

SEQ ID NO: 9 - protein, 281 aa, Ehrlichia chaffeensis
  amino acid sequence of E. chaffeensis P28
MNYKKVFITSALISLISSLPGVSFSDPAGSGINGNFYISGKYMPSASHFGVFSAKEERNTTVGVFGLKQNWDGSAISNSSPNDVFTVSNYSFKYENNPFLGFAGAIGYSMDGPRIELEVSYETFDVKNQGNNYKNEAHRYCALSHNSAADMSSASNNFVFLKNEGLLDISFMLNACYDVVGEGIPFSPYICAGIGTDLVSMFEATNPKISYQGKLGLSYSISPEASVFIGGHFHKVIGNEFRDIPTIIPTGSTLAGKGNYPAIVILDVCHFGIELGGRFAF

SEQ ID NO: 10 - protein, 283 aa, Ehrlichia chaffeensis
  amino acid sequence of E. chaffeensis OMP-1B
MNYKKIFVSSALISLMSILPYQSFADPVTSNDTGINDSREGFYISVKYNPSISHFRKFSAEEAPINGNTSITKKVFGLKKDGDIAQSANFNRTDPALEFQNNLISGFSGSIGYAMDGPRIELEAAYQKFDAKNPDNNDTNSGDYYKYFGLSREDAIADKKYVVLKNEGITFMSLMVNTCYDITAEGVPFIPYACAGVGADLINVFKDFNLKFSYQGKIGISYPITPEVSAFIGGYYHGVIGNNFNKIPVITPVVLEGAPQTTSALVTIDTGYFGGEVGVRFTF

SEQ ID NO: 11 - protein, 280 aa, Ehrlichia chaffeensis
  amino acid sequence of E. chaffeensis OMP-1C
MNCKKFFITTALALPMSFLPGILLSEPVQDDSVSGNFYISGKYMPSASHFGVFSAKEEKNPTVALYGLKQDWNGVSASSHADADFNNKGYSFKYENNPFLGFAGAIGYSMGGPRIEFEVSYETFDVKNQGGNYKNDAHRYCALDRKASSTNATASHYVLLKNEGLLDISLMLNACYDVVSEGIPFSPYICAGVGTDLISMFEAINPKISYQGKLGLSYSINPEASVFVGGHFHKVAGNEFRDISTLKAFATPSSAATPDLATVTLSVCHFGVELGGRFNF

SEQ ID NO: 12 - protein, 286 aa, Ehrlichia chaffeensis
  amino acid sequence of E. chaffeensis OMP-1D
MNCEKFFITTALTLLMSFLPGISLSDPVQDDNISGNFYISGKYMPSASHFGVFSAKEERNTTVGVFGIEQDWDRCVISRTTLSDIFTVPNYSFKYENNLFSGFAGAIGYSMDGPRIELEVSYEAFDVKNQGNNYKNEAHRYYALSHLLGTETQIDGAGSASVFLINEGLLDKSFMLNACYDVISEGIPFSPYICAGIGIDLVSMFEAINPKISYQGKLGLSYPISPEASVFIGGHFHKVIGNEFRDIPTMIPSESALAGKGNYPAIVTLDVFYFGIELGGRFNFQL

SEQ ID NO: 13 - protein, 278 aa, Ehrlichia chaffeensis
  amino acid sequence of E. chaffeensis OMP-1E
MNCKKFFITTALVSLMSFLPGISFSDPVQGDNISGNFYVSGKYMPSASHFGMFSAKEEKNPTVALYGLKQDWEGISSSSHNDNHFNNKGYSFKYENNPFLGFAGAIGYSMGGPRVEFEVSYETFDVKNQGNNYKNDAHRYCALGQQDNSGIPKTSKYVLLKSEGLLDISFMLNACYDIINESIPLSPYICAGVGTDLISMFEATNPKISYQGKLGLSYSINPEASVFIGGHFHKVIGNEFRDIPTLKAFVTSSATPDLAIVTLSVCHFGIELGGRFNF

SEQ ID NO: 14 - protein, 280 aa, Ehrlichia chaffeensis
  amino acid sequence of E. chaffeensis OMP-1F
MNCKKFFITTTLVSLMSFLPGISFSDAVQNDNVGGNFYISGKYVPSVSHFGVFSAKQERNTTTGVFGLKQDWDGSTISKNSPENTFNVPNYSFKYENNPFLGFAGAVGYLMNGPRIELEMSYETFDVKNQGNNYKNDAHKYYALTHNSGGKLSNAGDKFVFLKNEGLLDISLMLNACYDVISEGIPFSPYICAGVGTDLISMFEAINPKISYQGKLGLSYSISPEASVFVGGHFHKVIGNEFRDIPAMIPSTSTLTGNHFTIVTLSVCHFGVELGGRFNF

SEQ ID NO: 15 - protein, 284 aa, Cowdria ruminantium
  amino acid sequence of C. ruminantium MAP-1
MNCKKIFITSTLISLVSFLPGVSFSDVIQEENNPVGSVYISAKYMPTASHFGKMSIKEDSRDTKAVFGLKKDWDGVKTPSGNTNSIFTEKDYSFKYENNPFLGFAGAVGYSMNGPRIEFEVSYETFDVRNPGGNYKNDAHMYCALDTASSSTAGATTSVMVKNENLTDISLMLNACYDIMLDGMPVSPYVCAGIGTDLVSVINATNPKLSYQGKLGISYSINPEASIFIGGHFHRVIGNEFKDIATSKVFTSSGNASSAVSPGFASAILDVCHFGIEIGGRFVF

SEQ ID NO: 16 - DNA, 20 bp, artificial sequence
  primer_bind nucleotides 313-332 of C. ruminantium MAP-1,also nucleotides 307-326 of E. chaffeensis P28: forward primer 793 for PCR
gcaggagctgttggttactc

SEQ ID NO: 17 - DNA, 21 bp, artificial sequence
  primer_bind nucleotides 823-843 of C. ruminantium MAP-1,also nucleotides 814-834 of E. chaffeensis P28: reverse primer 1330 for PCR
ccttcctccaagttctatgcc

SEQ ID NO: 18 - DNA, 24 bp, artificial sequence
  primer_bind: primer 46f, specific for p28-5 gene
atatacttcctacctaatgtctca

SEQ ID NO: 19 - DNA, 20 bp, artificial sequence
  primer_bind: primer used for sequencing 28-kDa protein genes in E. canis
agtgcagagtcttcggtttc

SEQ ID NO: 20 - DNA, 18 bp, artificial sequence
  primer_bind: primer used for sequencing 28-kDa protein genes in E. canis
gttacttgcggaggacat

SEQ ID NO: 21 - DNA, 24 bp, artificial sequence
  primer_bind nucleotides 687-710 of E. canis p28-7: primer 394 for PCR
gcatttccacaggatcataggtaa

SEQ ID NO: 22 - DNA, 24 bp, artificial sequence
  primer_bind nucleotides 710-687 of E. canis p28-7: primer 394C for PCR
ttacctatgatcctgtggaaatgc

SEQ ID NO: 23 - DNA, 20 bp, artificial sequence
  primer_bind: primer 793C which anneals to a region with E. canis p28-7, used to amplify the intergenic region between gene p28-6 and p28-7
gagtaaccaacagctcctgc

SEQ ID NO: 24 - DNA, 24 bp, artificial sequence
  primer_bind: primer EC28OM-F complementary to noncoding regions adjacent to the open reading frame of p28-7
tctactttgcacttccactattgt

SEQ ID NO: 25 - DNA, 24 bp, artificial sequence
  primer_bind: primer EC28OM-R complementary to noncoding regions adjacent to the open reading frame of p28-7
attcttttgccactatttttcttt

SEQ ID NO: 26 - DNA, 25 bp, artificial sequence
  primer_bind: primer ECaSA3-2 corresponding to regions within p28-6, used to amplify the intergenic region NC3 between gene p28-6 and p28-7
ctaggattaggttatagtataagtt

SEQ ID NO: 27 - protein, 23 aa, Ehrlichia canis
  PEPTIDE: a predicted N-terminal signal peptide of p28-7 and p28-6
MNCKKILITTALMSLMYYAPSIS

SEQ ID NO: 28 - protein, 25 aa, Ehrlichia chaffeensis
  amino acid sequence of N-terminal signal peptide of E. chaffeensis P28
MNYKKILITSALISLISSLPGVSFS

SEQ ID NO: 29 - protein, 26 aa, Ehrlichia canis
  amino acid sequence of putative cleavage site of p28-7
MNCKKILITTALISLMYSIPSISSFS

SEQ ID NO: 30 - DNA, 299 bp, Ehrlichia canis
  nucleic acid sequence of intergenic noncoding region 1 (28NC1)
taatacttctattgtacatgttaaaaatagtactagtttgcttctgtggtttataaacgcaagagagaaatagttagtaataaattagaaagttaaatattagaaaagtcatatgtttttcattgtcattgatactcaactaaaagtagtataaatgttacttattaataattttacgtagtatattaaatttcccttacaaaagccactagtattttatactaaaagctatactttggcttgtatttaatttgtatttttactactgttaatttactttcactgtttctggtgtaaat

SEQ ID NO: 31 - DNA, 345 bp, Ehrlichia canis
  nucleic acid sequence of intergenic noncoding region 2 (28NC2)
taatttcgtggtacacatatcacgaagctaaaattgtttttttatctctgctgtatacaagagaaaaaatagtagtgaaaattacctaacaatatgacagtacaagtttaccaagcttattctcacaaaacttcttgtgtcttttatctctttacaatgaaatgtacacttagcttcactactgtagagtgtgtttatcaatgctttgtttattaatactctacataatatgttaaatttttcttacaaaactcactagtaatttatactagaatatatattctgacttgtatttgctttatacttccactattgttaatttattttcactattttaggtgtaat

SEQ ID NO: 32 - DNA, 345 bp, Ehrlichia canis
  nucleic acid sequence of intergenic noncoding region 3 (28NC3)
tgattttattgttgccacatattaaaaatgatctaaacttgtttttattattgctacatacaaaaaaaagaaaaatagtggcaaaagaatgtagcaataagaggggggggggggactaaatttaccttctattcttctaatattctttactatattcaaatagcacaactcaatgcttccaggaaaatatgtttctaatattttatttattaccaatccttatataatatattaaatttctcttacaaaaatctctaatgttttatacttaatatatatattctggcttgtatttactttgcacttccactattgttaatttattttcactattttaggtgtaat

SEQ ID NO: 33 - DNA, 355 bp, Ehrlichia canis
  nucleic acid sequence of intergenic noncoding region 4 (28NC4)
taattttattgttgccacatattaaaaatgatctaaacttgtttttawtattgctacatacaaaaaaagaaaaatagtggcaaaagaatgtagcaataagaggggggggggggaccaaatttatcttctatgcttcccaagttttttcycgctatttatgacttaaacaacagaaggtaatatcctcacggaaaacttatcttcaaatattttatttattaccaatcttatataatatattaaatttctcttacaaaaatcactagtattttataccaaaatatatattctgacttgcttttcttctgcacttctactatttttaatttatttgtcactattaggttataataaw

SEQ ID NO: 34 - DNA, 24 bp, artificial sequence
  primer p28-5-818f
ttaaacatatgccacttcggacta

SEQ ID NO: 35 - DNA, 28 bp, artificial sequence
  primer 1191
tatgatcgtgtaaaattgctgtgagtat

SEQ ID NO: 36 - DNA, 20 bp, artificial sequence
  primer ECa28-75C
tactggcacgtgctggacta

SEQ ID NO: 37 - DNA, 22 bp, artificial sequence
  primer ECa5′-1600
caccaataaatgcagagacttc

SEQ ID NO: 38 - DNA, 26 bp, artificial sequence
  primer 3125
aatccatcatttctcattacagtgtg

SEQ ID NO: 39 - DNA, 879 bp, Ehrlichia canis
  nucleic acid sequence of E. canis p28-1
atgaataataaactcaaatttactataataaacacagtattagtatgcttattgtcattacctaatatatcttcctcaaaggccataaacaataacgctaaaaagtactacggattatatatcagtggacaatataaacccagtgtttctgttttcagtaatttttcagttaaagaaaccaatgtcataactaaaaaccttatagctttaaaaaaagatgttgactctattgaaaccaagactgatgccagtgtaggtattagtaacccatcaaattttactatcccctatacagctgtatttcaagataattctgtcaatttcaatggaactattggttacacctttgctgaaggtacaagagttgaaatagaaggttcttatgaggaatttgatgttaaaaaccctggaggctatacactaagtgatgcctatcgctattttgcattagcacgtgaaatgaaaggtaatagttttacacctaaagaaaaagtttctaatagtatttttcacactgtaatgagaaatgatggattatctataatatctgttatagtaaatgtttgctacgatttctctttgaacaatttgtcaatatcgccttacatatgtggaggagcaggggtagatgctatagaattcttcgatgtattacacattaagtttgcatatcaaagcaagctaggtattgcttattctctaccatctaacattagtctctttgctagtttatattaccataaagtaatgggcaatcaatttaaaaatttaaatgtccaacatgttgctgaacttgcaagtatacctaaaattacatccgcagttgctacacttaatattggttattttggaggtgaaattggtgcaagattgacattt

SEQ ID NO: 40 - protein, 293 aa, Ehrlichia canis
  amino acid sequence of E. canis p28-1 protein
MNNKLKFTIINTVLVCLLSLPNISSSKAINNNAKKYYGLYISGQYKPSVSVFSNFSVKETNVITKNLIALKKDVDSIETKTDASVGISNPSNFTIPYTAVFQDNSVNFNGTIGYTFAEGTRVEIEGSYEEFDVKNPGGYTLSDAYRYFALAREMKGNSFTPKEKVSNSIFHTVMRNDGLSIISVIVNVCYDFSLNNLSISPYICGGAGVDAIEFFDVLHIKFAYQSKLGIAYSLPSNISLFASLYYHKVMGNQFKNLNVQHVAELASIPKITSAVATLNIGYFGGEIGARLTF

SEQ ID NO: 41 - DNA, 840 bp, Ehrlichia canis
  nucleic acid sequence of E. canis p28-2
atgaattataagaaaattctagtaagaagcgcgttaatctcattaatgtcaatcttaccatatcagtcttttgcagatcctgtaggttcaagaactaatgataacaaagaaggcttctacattagtgcaaagtacaatccaagtatatcacactttagaaaattctctgctgaagaaactcctattaatggaacaaattctctcactaaaaaagttttcggactaaagaaagatggtgatataacaaaaaaagacgattttacaagagtagctccaggcattgattttcaaaataacttaatatcaggattttcaggaagtattggttactctatggacggaccaagaatagaacttgaagctgcatatcaacaatttaatccaaaaaacaccgataacaatgatactgataatggtgaatactataaacattttgcattatctcgtaaagatgcaatggaagatcagcaatatgtagtacttaaaaatgacggcataacttttatgtcattgatggttaatacttgctatgacattacagctgaaggagtatctttcgtaccatatgcatgtgcaggtataggagcagatcttatcactatttttaaagacctcaatctaaaatttgcttaccaaggaaaaataggtattagttaccctatcacaccagaagtctctgcatttattggtggatactaccatggcgttattggtaataaatttgagaagatacctgtaataactcctgtagtattaaatgatgctcctcaaaccacatctgcttcagtaactcttgacgttggatactttggcggagaaattggaatgaggttcaccttc

SEQ ID NO: 42 - protein, 280 aa, Ehrlichia canis
  amino acid sequence of E. canis p28-2 protein
MNYKKILVRSALISLMSILPYQSFADPVGSRTNDNKEGFYISAKYNPSISHFRKFSAEETPINGTNSLTKKVFGLKKDGDITKKDDFTRVAPGIDFQNNLISGFSGSIGYSMDGPRIELEAAYQQFNPKNTDNNDTDNGEYYKHFALSRKDAMEDQQYVVLKNDGITFMSLMVNTCYDITAEGVSFVPYACAGIGADLITIFKDLNLKFAYQGKIGISYPITPEVSAFIGGYYHGVIGNKFEKIPVITPVVLNDAPQTTSASVTLDVGYFGGEIGMRFTF

SEQ ID NO: 43 - DNA, 828 bp, Ehrlichia canis
  nucleic acid sequence of E. canis p28-3
atgaactgtaaaaaaattcttataacaactacattggtatcactaacaattcttttacctggcatatctttctccaaaccaatacatgaaaacaatactacaggaaacttttacattattggaaaatatgtaccaagtatttcacattttgggaacttttcagctaaagaagaaaaaaacacaacaactggaatttttggattaaaagaatcatggactggtggtatcatccttgataaagaacatgcagcttttaatatcccaaattattcatttaaatatgaaaataatccatttttaggatttgcaggggtaattggctattcaataggtagtccaagaatagaatttgaagtatcatacgagacattcgatgtacaaaatccaggagataagtttaacaatgatgcacataagtattgtgctttatccaatgattccagtaaaacaatgaaaagtggtaaattcgtttttctcaaaaatgaaggattaagtgacatatcactcatgttaaatgtatgttatgatataataaacaaaagaatgcctttttcaccttacatatgtgcaggcattggtactgacttaatattcatgtttgacgctataaaccataaagctgcttatcaaggaaaattaggttttaattatccaataagcccagaagctaacatttctatgggtgtgcactttcacaaagtaacaaacaacgagtttagagttcctgttctattaactgctggaggactcgctccagataatctatttgcaatagtaaagttgagtatatgtcattttgggttagaatttgggtacagggtcagtttt

SEQ ID NO: 44 - protein, 276 aa, Ehrlichia canis
  amino acid sequence of E. canis p28-3 protein
MNCKKILITTTLVSLTILLPGISFSKPIHENNTTGNFYIIGKYVPSISHFGNFSAKEEKNTTTGIFGLKESWTGGIILDKEHAAFNIPNYSFKYENNPFLGFAGVIGYSIGSPRIEFEVSYETFDVQNPGDKFNNDAHKYCALSNDSSKTMKSGKFVFLKNEGLSDISLMLNVCYDIINKRMPFSPYICAGIGTDLIFMFDAINHKAAYQGKLGFNYPISPEANISMGVHFHKVTNNEFRVPVLLTAGGLAPDNLFAIVKLSICHFGLEFGYRVSF

SEQ ID NO: 45 - DNA, 813 bp, Ehrlichia canis
  nucleic acid sequence of E. canis p28-9
atgaattacaaaagatttgttgtaggtgttacgctgagtacatttgtttttttcttatctgatggtgctttttctgatgcaaatttttctgaagggaggagaggactttatataggtagtcagtataaagttggtattcccaattttagtaatttttcagctgaagaaacaattcctggtattacaaaaaagatttttgcgttaggtcttgataagtctgagataaatactcacagcaattttacacgatcatatgaccctacttatgcaagcagttttgcagggtttagtggtatcattggatattatgttaatgactttagggtagaatttgaaggttcttatgagaattttgaacctgaaagacaatggtaccctgagaatagccaaagctacaaattttttgctttgtctcgaaatgctacaaatagtgataataagtttatagtactagagaataacggcgttgttgacaagtctcttaatgtaaatgtttgttatgatattgctagtggtagtattcctttagcaccttatatgtgtgctggtgttggtgcagattatataaagtttttaggtatatcattgcctaagttttcttatcaagttaagtttggtgtcaactaccctctaaatgttaatactatgttgtttggtgggggttattaccataaggttgtaggtgataggcatgagagagtagaaatagcttaccatcctactgcattatctgacgttcctagaactacttcagcttctgctactttaaatactgattattttggttgggagattggatttagatttgcgcta

SEQ ID NO: 46 - protein, 271 aa, Ehrlichia canis
  amino acid sequence of E. canis p28-9 protein
MNYKRFVVGVTLSTFVFFLSDGAFSDANFSEGRRGLYIGSQYKVGIPNFSNFSAEETIPGITKKIFALGLDKSEINTHSNFTRSYDPTYASSFAGFSGIIGYYVNDFRVEFEGSYENFEPERQWYPENSQSYKFFALSRNATNSDNKFIVLENNGVVDKSLNVNVCYDIASGSIPLAPYMCAGVGADYIKFLGISLPKFSYQVKFGVNYPLNVNTMLFGGGYYHKVVGDRHERVEIAYHPTALSDVPRTTSASATLNTDYFGWEIGFRFAL

(19) United States
(12) Patent Application Publication
(10) Pub. No.: US 2004/0198951 A1
(43) Pub. Date: Oct. 7, 2004

(54) Homologous 28-kilodalton immunodominant protein genes of Ehrlichia canis and uses thereof

(75) Inventors: David H. Walker, Galveston, TX (US); Xue-Jie Yu, Houston, TX (US); Jere W. McBride, Galveston, TX (US)

Correspondence Address: Benjamin Aaron Adler, Ph.D., J.D., Adler & Associates, 8011 Candle Lane, Houston, TX, 77071, US

(73) Assignee: Research Development Foundation

(21) Appl. No.: 10/680,349

(22) Filed: Oct. 7, 2003

Related U.S. Application Data
(62) Division of application No. 10/062,624, filed on Jan. 31, 2002, now abandoned.
Application No. 10/062,624 is a division of application No. 09/660,587, filed on Sep. 12, 2000, now Pat. No. 6,392,023.
Application No. 09/660,587 is a continuation-in-part of application No. 09/261,358, filed on Mar. 3, 1999, now Pat. No. 6,403,780.
Application No. 09/261,358 is a continuation-in-part of application No. 09/201,458, filed on Nov. 30, 1998, now Pat. No. 6,458,942.

(51) Int. Cl. C07K 1/00; C07K 14/00; C07K 17/00

(52) U.S. Cl. 530/350

(57) ABSTRACT

The present invention is directed to the cloning, sequencing and expression of homologous immunoreactive 28-kDa protein genes, p28-1, -2, -3, -5, -6, -7, -9, from a polymorphic multiple gene family of Ehrlichia canis. Further disclosed is a multigene locus encoding all nine homologous 28-kDa protein genes of Ehrlichia canis. Recombinant Ehrlichia canis 28-kDa proteins react with convalescent phase antiserum from an E. canis-infected dog, and may be useful in the development of vaccines and serodiagnostics that are particularly effective for disease prevention and serodiagnosis.

### CROSS-REFERENCE TO RELATED APPLICATION

This patent application is a divisional application of U.S. Ser. No. 10/062,624, filed Jan. 31, 2002, which is a divisional of U.S. Ser. No. 09/660,587, filed Sep. 12, 2000, now issued as U.S. Pat. No. 6,392,023, which is a continuation-in-part of U.S. Ser. No. 09/261,358, filed Mar. 3, 1999, now issued as U.S. Pat. No. 6,403,780, which is a continuation-in-part of U.S. Ser. No. 09/201,458, filed Nov. 30, 1998, now issued as U.S. Pat. No. 6,458,942.

### BACKGROUND OF THE INVENTION

1. Field of the Invention

The present invention relates generally to the field of molecular biology. More specifically, the present invention relates to molecular cloning and characterization of homologous 28-kDa protein genes in Ehrlichia canis, a multigene locus encoding the 28-kDa homologous proteins of Ehrlichia canis and uses thereof.

2. Description of the Related Art

Canine ehrlichiosis, also known as canine tropical pancytopenia, is a tick-borne rickettsial disease of dogs first described in Africa in 1935 and the United States in 1963 (Donatien and Lestoquard, 1935; Ewing, 1963). The disease became better recognized after an epizootic outbreak occurred in United States military dogs during the Vietnam War (Walker et al., 1970)

The etiologic agent of canine ehrlichiosis is Ehrlichia canis, a small, gram-negative, obligate intracellular bacterium which exhibits tropism for mononuclear phagocytes (Nyindo et al., 1971) and is transmitted by the brown dog tick, Rhipicephalus sanguineus (Groves et al., 1975). The progression of canine ehrlichiosis occurs in three phases, acute, subclinical and chronic. The acute phase is characterized by fever, anorexia, depression, lymphadenopathy and mild thrombocytopenia (Troy and Forrester, 1990). Dogs typically recover from the acute phase, but become persistently infected carriers of the organism without clinical signs of disease for months or even years (Harrus et al., 1998). A chronic phase develops in some cases that is characterized by thrombocytopenia, hyperglobulinemia, anorexia, emaciation, and hemorrhage, particularly epistaxis, followed by death (Troy and Forrester, 1990).

Regulation of surface antigenicity may be an important mechanism for the establishment of such persistent infections in the host. Although disease pathogenesis is poorly understood, multigene families described in members of the related genera Ehrlichia, Anaplasma, and Cowdria may be involved in variation of major surface antigen expression thereby evading immune surveillance. Anaplasma marginale, an organism closely related to E. canis, exhibits variation of major surface protein 3 (msp-3) genes resulting in antigenic polymorphism among strains (Alleman et al., 1997).

Molecular taxonomic analysis based on the 16S rRNA gene has determined that E. canis and E. chaffeensis, the etiologic agent of human monocytic ehrlichiosis (HME), are closely related (Anderson et al., 1991; Anderson et al., 1992; Dawson et al., 1991; Chen et al., 1994). Considerable cross reactivity of the 64, 47, 40, 30, 29 and 23-kDa antigens between E. canis and E. chaffeensis has been reported (Chen et al., 1994; Chen et al., 1997; Rikihisa et al., 1994; Rikihisa et al., 1992). Analysis of immunoreactive antigens with human and canine convalescent phase sera by immunoblot has resulted in the identification of numerous immunodominant proteins of E. canis, including a 30-kDa protein (Chen et al., 1997). In addition, a 30-kDa protein of E. canis has been described as a major immunodominant antigen recognized early in the immune response that is antigenically distinct from the 30-kDa protein of E. chaffeensis (Rikihisa et al., 1992; Rikihisa et al., 1994). Other immunodominant proteins of E. canis with molecular masses ranging from 20 to 30-kDa have also been identified (Brouqui et al., 1992; Nyindo et al., 1991; Chen et al., 1994; Chen et al., 1997).

Homologous 28-32 kDa immunodominant proteins encoded by multigene families have been reported in related organisms including, E. chaffeensis and Cowdria ruminantium (Sulsona et al., 1999; Ohashi et al., 1998a; Reddy et al., 1998). Recently, characterization of a 21 member multigene family encoding proteins of 23 to 28-kDa has been described in E. chaffeensis (Yu et al., 2000). The E. chaffeensis 28-kDa outer membrane proteins are surface exposed, and contain three major hypervariable regions (Ohashi et al., 1998a). The recombinant E. chaffeensis P28 appeared to provide protection against homologous challenge infection in mice, and antisera produced against the recombinant protein cross reacted with a 30-kDa protein of E. canis (Ohashi et al., 1998a). Diversity in the p28 gene among E. chaffeensis isolates has been reported (Yu et al., 1999a), and studies using monoclonal antibodies have further demonstrated diversity in the expressed P28 proteins (Yu et al., 1993). Conversely, complete conservation of a p28 genes in geographically different isolates of E. canis has been reported and suggests that E. canis may be conserved in North America (McBride et al., 1999, 2000).

The prior art is deficient in the lack of cloning and characterization of new homologous 28-kDa immunoreactive protein genes of Ehrlichia canis and a single multigene locus containing the homologous 28-kDa protein genes. Further, The prior art is deficient in the lack of recombinant proteins of such immunoreactive genes of Ehrlichia canis. The present invention fulfills this long-standing need and desire in the art.

### SUMMARY OF THE INVENTION

Certain embodiments of the present invention describe the molecular cloning, sequencing, characterization, and expression of homologous mature 28-kDa immunoreactive protein genes of Ehrlichia canis (designated p28-1, -2, -3, -5, -6, -7, -9), and the identification of a single locus (10,677-bp) containing nine 28-kDa protein genes of Ehrlichia canis (p28-1 to p28-9). Eight of the p28 genes were located on one DNA strand, and one p28 gene was found on the complementary strand. The nucleic acid homology among the nine p28 gene members was 37 to 75%, and the amino acid homology ranged from 28 to 72%.

In one embodiment of the present invention, there are provided DNA sequences encoding a 30-kDa immunoreactive protein of Ehrlichia canis. Preferably, the protein has an amino acid sequence selected from the group consisting of SEQ ID No. 2, 4, 6, 40, 42, 44, 46 and the gene has a nucleic acid sequence selected from the group consisting of SEQ ID No. 1, 3, 5, 39, 41, 43, 45 and is a member of a polymorphic multiple gene family. Generally, the protein has an N-terminal signal sequence which may be cleaved after post-translational process resulting in the production of a mature 28-kDa protein. Furthermore, the genes encoding 28-kDa proteins are preferably contained in a single multigene locus, which has the size of 10,677 bp and encodes nine homologous 28-kDa proteins of Ehrlichia canis.

In another embodiment of the present invention, there is provided an expression vector comprising a gene encoding a 28-kDa immunoreactive protein of Ehrlichia canis and capable of expressing the gene when the vector is introduced into a cell.

In still another embodiment of the present invention, there is provided a recombinant protein comprising an amino acid sequence selected from the group consisting of SEQ ID No. 2, 4, 6, 40, 42, 44, and 46. Preferably, the amino acid sequence is encoded by a nucleic acid sequence selected from the group consisting of SEQ ID No. 1, 3, 5, 39, 41, 43, and 45. Preferably, the recombinant protein comprises four variable regions which may be surface exposed, hydrophilic and antigenic. The recombinant protein may be useful as an antigen.

In yet another embodiment of the present invention, there is provided a method of producing the recombinant protein, comprising the steps of obtaining a vector that comprises an expression region comprising a sequence encoding the amino acid sequence selected from the group consisting of SEQ ID No. 2, 4, 6, 40, 42, 44, and 46 operatively linked to a promoter; transfecting the vector into a cell; and culturing the cell under conditions effective for expression of the expression region.

The invention may also be described in certain embodiments as a method of inhibiting Ehrlichia canis infection in a subject comprising the steps of: identifying a subject prior to exposure or suspected of being exposed to or infected with Ehrlichia canis; and administering a composition comprising a 28-kDa antigen of Ehrlichia canis in an amount effective to inhibit an Ehrlichia canis infection. The inhibition may occur through any means such as, e.g., the stimulation of the subject's humoral or cellular immune responses, or by other means such as inhibiting the normal function of the 28-kDa antigen, or even competing with the antigen for interaction with some agent in the subject's body.

Other and further aspects, features, and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions of the invention briefly summarized above may be had by reference to certain embodiments thereof which are illustrated in the appended drawings. These drawings form a part of the specification. It is to be noted, however, that the appended drawings illustrate preferred embodiments of the invention and therefore are not to be considered limiting in their scope.

FIG. 1 shows nucleic acid sequence (SEQ ID No. 1) and deduced amino acid sequence (SEQ ID No. 2) of p28-7 gene including adjacent 5′ and 3′ non-coding sequences. The ATG start codon and TAA termination are shown in bold, and the 23 amino acid leader signal sequence is underlined.

FIG. 2 shows SDS-PAGE of expressed 50-kDa recombinant p28-7-thioredoxin fusion protein (Lane 1, arrow) and 16-kDa thioredoxin control (Lane 2, arrow), and corresponding immunoblot of recombinant p28-7-thioredoxin fusion protein recognized by covalescent-phase E. canis canine antiserum (Lane 3). Thiroredoxin control was not detected by E. canis antiserum (not shown).

FIG. 3 shows amino acid sequences alignment of p28-7 protein (ECa28-1, SEQ ID NO. 2), p28-5 protein (ECa28SA2, partial sequence, SEQ ID NO. 7), p28-4 protein (ECa28SA1, SEQ ID NO. 8), E. chaffeensis P28 (SEQ ID NO. 9), E. chaffeensis OMP-1 family (SEQ ID NOs: 10-14) and C. ruminantium MAP-1 protein (SEQ ID NO. 15). The p28-7 amino acid sequence is presented as the consensus sequence. Amino acids not shown are identical to p28-7 and are represented by a dot. Divergent amino acids are shown with the corresponding one letter abbreviation. Gaps introduced for maximal alignment of the amino acid sequences are denoted with a dash. Variable regions are underlined and denoted (VR1, VR2, VR3, and VR4). The arrows indicate the predicted signal peptidase cleavage site for the signal peptide.

FIG. 4 shows phylogenetic relatedness of E. canis p28-7 (ECa28-1), p28-5 (ECa28SA2, partial sequence), p28-4 (ECa28SA1), members of the E. chaffeensis omp-1 multiple gene family, and C. rumanintium map-1 protein from deduced amino acid sequences utilizing unbalanced tree construction. The length of each pair of branches represents the distance between the amino acid sequence of the pairs. The scale measures the distance between sequences.

FIG. 5 shows Southern blot analysis of E. canis genomic DNA completely digested with six individual restriction enzymes and hybridized with a p28-7 DIG-labeled probe (Lanes 2-7); DIG-labeled molecular weight markers (Lanes 1 and 8).

FIG. 6 shows comparison of predicted protein characteristics of E. canis p28-7 (ECa28-1, Jake strain) and E. chaffeensis P28 (Arkansas strain). Surface probability predicts the surface residues by using a window of hexapeptide. A surface residue is any residue with a >2.0 nm² of water accessible surface area. A hexapeptide with a value higher than 1 was considered as surface region. The antigenic index predicts potential antigenic determinants. The regions with a value above zero are potential antigenic determinants. T-cell motif locates the potential T-cell antigenic determinants by using a motif of 5 amino acids with residue 1-glycine or polar, residue 2-hydrophobic, residue 3-hydrophobic, residue 4-hydrophobic or proline, and residue 5-polar or glycine. The scale indicates amino acid positions.

FIG. 7 shows nucleic acid sequences and deduced amino acid sequences of the E. canis 28-kDa protein genes p28-5 (nucleotide 1-849: SEQ ID No. 3; amino acid sequence: SEQ ID No. 4) and p28-6 (nucleotide 1195-2031: SEQ ID No. 5; amino acid sequence: SEQ ID No. 6) including intergenic noncoding sequences (NC2, nucleotide 850-1194: SEQ ID No. 31). The ATG start codon and termination condons are shown in bold.

FIG. 8 shows schematic of the E. canis 28-kDa protein gene locus (5.592-Kb, containing five genes) indicating genomic orientation and intergenic noncoding regions (28NC1-4). The 28-kDa protein genes shown in Locus 1 and 2 (shaded) have been described (McBride et al., 1999; Reddy et al., 1998; Ohashi et al., 1998). The complete sequence of p28-5 and a new 28-kDa protein gene designated p28-6 was sequenced. The noncoding intergenic regions (28NC2-3) between p28-5, p28-6 and p28-7 were completed joining the previously unlinked loci 1 and 2.

FIG. 9 shows phylogenetic relatedness of the E. canis 28-kDa protein gene p28-4 (ECa28SA1), p28-5 (ECa28SA2), p28-6 (ECa28SA3), p28-7 (ECa28-1) and p28-8 (ECa28-2) based on amino acid sequences utilizing unbalanced tree construction. The length of each pair of branches represents the distance between amino acid pairs. The scale measures the distance between sequences.

FIG. 10 shows alignment of E. canis 28-kDa protein gene intergenic noncoding nucleic acid sequences (SEQ ID Nos. 30-33). Nucleic acids not shown, denoted with a dot (.), are identical to noncoding region 1 (28NC1). Divergence is shown with the corresponding one letter abbreviation. Gaps introduced for maximal alignment of the amino acid sequences are denoted with a dash (-). Putative transcriptional promoter regions (−10 and −35) and ribosomal binding site (RBS) are boxed.

FIG. 11 shows schematic representation of the nine gene E. canis p28 locus (10,677-bp) indicating genomic orientation and intergenic noncoding regions. The p28 genes (p28-1, 2, 3, 9) (unshaded) were identified in Example 8. Shaded p28 genes have been identified previously and designated as follows: p28-4, p30a (Ohashi et al., 1998b) and ORF1 (Reddy et al., 1998); p28-5 and p28-6, (McBride, et. al., 2000); p28-7, p28 (McBride et al., 1999) and p30 (Ohashi et al., 1998b); and p28-8, p30-1 (Ohashi et al., 1998b).

FIG. 12 shows phylogenetic relationships of E. canis P28-1 to P28-9 based on the amino acid sequences. The length of each pair of branches represents the distance between amino acid pairs. The scale measures the percentage of divergence between the sequences.

FIG. 13 shows nucleic acid sequence (SEQ ID No. 39) and deduced amino acid sequence (SEQ ID No. 40) of E. canis p28-1 gene.

FIG. 14 shows nucleic acid sequence (SEQ ID No. 41) and deduced amino acid sequence (SEQ ID No. 42) of E. canis p28-2 gene.

FIG. 15 shows nucleic acid sequence (SEQ ID No. 43) and deduced amino acid sequence (SEQ ID No. 44) of E. canis p28-3 gene.

FIG. 16 shows nucleic acid sequence (SEQ ID No. 45) and deduced amino acid sequence (SEQ ID No. 46) of E. canis p28-9 gene.

### DETAILED DESCRIPTION OF THE INVENTION

The present invention describes cloning, sequencing and expression of homologous genes encoding a 30-kilodalton (kDa) protein of Ehrlichia canis. A comparative molecular analysis of homologous genes among seven E. canis isolates and the E. chaffeensis omp-1 multigene family was also performed. Several new 28-kDa protein genes are identified as follows:

p28-7 (ECa28-1) has an 834-bp open reading frame encoding a protein of 278 amino acids (SEQ ID No. 2) with a predicted molecular mass of 30.5-kDa. An N-terminal signal sequence was identified suggesting that the protein is post-translationally modified to a mature protein of 27.7-kDa.

P28-6 (ECa28SA3) has an 840-bp open reading frame encoding a 280 amino acid protein (SEQ ID No. 6).

Using PCR to amplify 28-kDa protein genes of E. canis, a previously unsequenced region of p28-5 (Eca28SA2) was completed. Sequence analysis of p28-5 revealed an 849-bp open reading frame encoding a 283 amino acid protein (SEQ ID No. 4).

PCR amplification using primers specific for 28-kDa protein gene intergenic noncoding regions led to the sequencing of regions linkeding two previously separate loci, thereby identifying a single locus (5.592-kb) containing five 28-kDa protein genes (p28-4, -5, -6, -7 and -8). The five 28-kDa proteins were predicted to have signal peptides resulting in mature proteins, and had amino acid homology ranging from 51 to 72%. Analysis of intergenic regions revealed hypothetical promoter regions for each gene, suggesting that these genes may be independently and differentially expressed. Intergenic noncoding regions (28NC1-4) ranged in size from 299 to 355-bp, and were 48 to 71% homologous.

Furthermore, previously unknown regions of DNA upstream and downstream of the above five gene locus of tandemly arranged p28 genes were sequenced, and p28-1, -2, -3, and -9 were identified. Consequently, a nine gene E. canis p28 locus spanning 10, 677 bp was identified in the present invention.

The present invention is directed to, inter alia, homologous 28-kDa protein genes in Ehrlichia canis, p28-1, -2, -3, -6, -7, and p28-9, and a complete sequence of previously partially sequenced p28-5. Also disclosed is a multigene locus encoding nine homologous 28-kDa outer membrane proteins of Ehrlichia canis. Eight of the p28 genes were located on one DNA strand, and one p28 gene was found on the complementary strand. The nucleic acid homology among the nine p28 gene members was 37 to 75%, and the amino acid homology ranged from 28 to 72%.

In accordance with the present invention there may be employed conventional molecular biology, microbiology, and recombinant DNA techniques within the skill of the art. Such techniques are explained fully in the literature. See, e.g., Maniatis, Fritsch & Sambrook, “Molecular Cloning: A Laboratory Manual (1982); “DNA Cloning: A Practical Approach,” Volumes I and II (D. N. Glover ed. 1985); “Oligonucleotide Synthesis” (M. J. Gait ed. 1984); “Nucleic Acid Hybridization” [B. D. Hames & S. J. Higgins eds. (1985)]; “Transcription and Translation” [B. D. Hames & S. J. Higgins eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

The invention includes a substantially pure DNA encoding a 28-kDa immunoreactive protein of Ehrlichia canis. The protein encoded by the DNA of this invention may share at least 80% sequence identity (preferably 85%, more preferably 90%, and most preferably 95%) with the amino acids listed in SEQ ID No. 2, 4, 6, 40, 42, 44 or 46. More preferably, the DNA includes the coding sequence of the nucleotides of SEQ ID No. 1, 3, 5, 39, 41, 43, 45, or a degenerate variant of such a sequence.

It is well known in the art that the amino acid sequence of a protein is determined by the nucleotide sequence of the DNA that encodes the protein. Because of the degeneracy of the genetic code (i.e., for most amino acids, more than one nucleotide triplet (codon) codes for a single amino acid), different nucleotide sequences can code for a particular amino acid, or polypeptide. Thus, the polynucleotide sequences of the subject invention also encompass those degenerate sequences that encode the polypeptides of the subject invention, or a fragment or variant thereof.

This invention also includes a substantially pure DNA containing a sequence of at least 15 consecutive nucleotides (preferably 20, more preferably 30, even more preferably 50, and most preferably all) of the region from the nucleotides listed in SEQ ID No 1, 3, 5, 39, 41, 43, or 45.

By “substantially pure DNA” is meant DNA that is not part of a milieu in which the DNA naturally occurs, by virtue of separation (partial or total purification) of some or all of the molecules of that milieu, or by virtue of alteration of sequences that flank the claimed DNA. The term therefore includes, for example, a recombinant DNA which is incorporated into a vector, into an autonomously replicating plasmid or virus, or into the genomic DNA of a prokaryote or eukaryote; or which exists as a separate molecule (e.g., a cDNA or a genomic or cDNA fragment produced by polymerase chain reaction (PCR) or restriction endonuclease digestion) independent of other sequences. It also includes a recombinant DNA which is part of a hybrid gene encoding an additional polypeptide sequence, e.g., a fusion protein. Also included in the present invention is a recombinant DNA which includes a portion of the nucleotides listed in SEQ ID No 1, 3, 5, 39, 41, 43, or 45 which encodes a 28-kDa immunoreactive protein of Ehrlichia canis.

The DNA should have at least about 70% sequence identity to the coding sequence of the nucleotides listed in SEQ ID No 1, 3, 5, 39, 41, 43, or 45, preferably at least 75% (e.g. at least 80%); and most preferably at least 90% identity. The identity between two sequences is a direct function of the number of matching or identical positions. When a subunit position in both of the two sequences is occupied by the same monomeric subunit, e.g., if a given position is occupied by an adenine in each of two DNA molecules, then they are identical at that position. For example, if 7 positions in a sequence 10 nucleotides in length are identical to the corresponding positions in a second 10-nucleotide sequence, then the two sequences have 70% sequence identity. The length of comparison sequences will generally be at least 50 nucleotides, preferably at least 60 nucleotides, more preferably at least 75 nucleotides, and most preferably 100 nucleotides. Sequence identity is typically measured using sequence analysis software (e.g., Sequence Analysis Software Package of the Genetics Computer Group, University of Wisconsin Biotechnology Center, 1710 University Avenue, Madison, Wis. 53705).

The present invention also comprises a vector comprising a DNA sequence coding for a which encodes a gene encoding a 28-kDa immunoreactive protein of Ehrlichia canis and said vector is capable of replication in a host which comprises, in operable linkage: a) an origin of replication; b) a promoter; and c) a DNA sequence coding for said protein. Preferably, the vector of the present invention contains a portion of the DNA sequence shown in SEQ ID No 1, 3, 5, 39, 41, 43, or 45.

A “vector” may be defined as a replicable nucleic acid construct, e.g., a plasmid or viral nucleic acid. Vectors may be used to amplify and/or express nucleic acid encoding a 28-kDa immunoreactive protein of Ehrlichia canis. An expression vector is a replicable construct in which a nucleic acid sequence encoding a polypeptide is operably linked to suitable control sequences capable of effecting expression of the polypeptide in a cell. The need for such control sequences will vary depending upon the cell selected and the transformation method chosen. Generally, control sequences include a transcriptional promoter and/or enhancer, suitable mRNA ribosomal binding sites, and sequences which control the termination of transcription and translation. Methods which are well known to those skilled in the art can be used to construct expression vectors containing appropriate transcriptional and translational control signals. See for example, the techniques described in Sambrook et al., 1989, Molecular Cloning: A Laboratory Manual (2nd Ed.), Cold Spring Harbor Press, N.Y. A gene and its transcription control sequences are defined as being “operably linked” if the transcription control sequences effectively control the transcription of the gene. Vectors of the invention include, but are not limited to, plasmid vectors and viral vectors. Preferred viral vectors of the invention are those derived from retroviruses, adenovirus, adeno-associated virus, SV40 virus, or herpes viruses.

In general, expression vectors containing promoter sequences which facilitate the efficient transcription of the inserted DNA fragment are used in connection with the host. As used herein, the term “host” is meant to include not only prokaryotes but also eukaryotes such as yeast, plant and animal cells. A recombinant DNA molecule or gene which encodes a 28-kDa immunoreactive protein of Ehrlichia canis of the present invention can be used to transform a host using any of the techniques commonly known to those of ordinary skill in the art. Especially preferred is the use of a vector containing coding sequences for a gene encoding a 28-kDa immunoreactive protein of Ehrlichia canis of the present invention for purposes of prokaryote transformation.

Prokaryotic hosts may include E. coli, S. tymphimurium, Serratia marcescens and Bacillus subtilis. Eukaryotic hosts include yeasts such as Pichia pastoris, mammalian cells and insect cells. The transformed hosts can be fermented and cultured according to means known in the art to achieve optimal cell growth.

As used herein, the term “engineered” or “recombinant” cell is intended to refer to a cell into which a recombinant gene, such as a gene encoding an Ehrlichia canis antigen has been introduced. Therefore, engineered cells are distinguishable from naturally occurring cells which do not contain a recombinantly introduced gene. Engineered cells are thus cells having a gene or genes introduced through the hand of man. Recombinantly introduced genes will either be in the form of a cDNA gene, a copy of a genomic gene, or will include genes positioned adjacent to a promoter not naturally associated with the particular introduced gene. In addition, the recombinant gene may be integrated into the host genome, or it may be contained in a vector, or in a bacterial genome transfected into the host cell.

The present invention is also drawn to substantially pure 28-30 kDa immunoreactive proteins of E. canis comprise of amino acid sequences listed in, for example, SEQ ID No. 2, 4, 6, 40, 42, 44, or 46.

By a “substantially pure protein” is meant a protein which has been separated from at least some of those components which naturally accompany it. Typically, the protein is substantially pure when it is at least 60%, by weight, free from the proteins and other naturally-occurring organic molecules with which it is naturally associated in vivo. Preferably, the purity of the preparation is at least 75%, more preferably at least 90%, and most preferably at least 99%, by weight. A substantially pure 28-kDa immunoreactive protein of Ehrlichia canis may be obtained, for example, by extraction from a natural source; by expression of a recombinant nucleic acid encoding a 28-kDa immunoreactive protein of Ehrlichia canis; or by chemically synthesizing the protein. Purity can be measured by any appropriate method, e.g., column chromatography such as immunoaffinity chromatography using an antibody specific for a 28-kDa immunoreactive protein of Ehrlichia canis, polyacrylamide gel electrophoresis, or HPLC analysis. A protein is substantially free of naturally associated components when it is separated from at least some of those contaminants which accompany it in its natural state. Thus, a protein which is chemically synthesized or produced in a cellular system different from the cell from which it naturally originates will be, by definition, substantially free from its naturally associated components. Accordingly, substantially pure proteins include eukaryotic proteins synthesized in E. coli, other prokaryotes, or any other organism in which they do not naturally occur.

In addition to substantially full-length proteins, the invention also includes fragments (e.g., antigenic fragments) of the 28-kDa immunoreactive protein of Ehrlichia canis (SEQ ID No. 2, 4, 6, 40, 42, 44, or 46). As used herein, “fragment,” as applied to a polypeptide, will ordinarily be at least 10 residues, more typically at least 20 residues, and preferably at least 30 (e.g., 50) residues in length, but less than the entire, intact sequence. Fragments of the 28-kDa immunoreactive protein of Ehrlichia canis can be generated by methods known to those skilled in the art, e.g., by enzymatic digestion of naturally occurring or recombinant 28-kDa immunoreactive protein of Ehrlichia canis, by recombinant DNA techniques using an expression vector that encodes a defined fragment of 28-kDa immunoreactive protein of Ehrlichia canis, or by chemical synthesis. The ability of a candidate fragment to exhibit a characteristic of 28-kDa immunoreactive protein of Ehrlichia canis (e.g., binding to an antibody specific for 28-kDa immunoreactive protein of Ehrlichia canis) can be assessed by methods described herein.

Purified 28-kDa immunoreactive protein of Ehrlichia canis or antigenic fragments of 28-kDa immunoreactive protein of Ehrlichia canis can be used to generate new antibodies or to test existing antibodies (e.g., as positive controls in a diagnostic assay) by employing standard protocols known to those skilled in the art.

As is well known in the art, a given polypeptide may vary in its immunogenicity. It is often necessary therefore to couple the immunogen (e.g., a polypeptide of the present invention) with a carrier. Exemplary and preferred carriers are keyhole limpet hemocyanin (KLH) and human serum albumin. Means for conjugating a polypeptide to a carrier protein are well known in the art and include glutaraldehyde, m-maleimidobenzoyl-N-hydroxysuccinimide ester, carbo-diimide and bis-biazotized benzidine. It is also understood that the peptide may be conjugated to a protein by genetic engineering techniques that are well known in the art.

As is also well known in the art, immunogenicity to a particular immunogen can be enhanced by the use of non-specific stimulators of the immune response known as adjuvants. Exemplary and preferred adjuvants include complete BCG, Detox, (RIBI, Immunochem Research Inc.) ISCOMS and aluminum hydroxide adjuvant (Superphos, Biosector).

Included in this invention are polyclonal antisera generated by using 28-kDa immunoreactive protein of Ehrlichia canis or a fragment of 28-kDa immunoreactive protein of Ehrlichia canis as the immunogen in, e.g., rabbits. Standard protocols for monoclonal and polyclonal antibody production known to those skilled in this art are employed. The monoclonal antibodies generated by this procedure can be screened for the ability to identify recombinant Ehrlichia canis cDNA clones, and to distinguish them from known cDNA clones.

The invention encompasses not only an intact monoclonal antibody, but also an immunologically-active antibody fragment, e.g., a Fab or (Fab)₂ fragment; an engineered single chain Fv molecule; or a chimeric molecule, e.g., an antibody which contains the binding specificity of one antibody, e.g., of murine origin, and the remaining portions of another antibody, e.g., of human origin.

In one embodiment, the antibody, or fragment thereof, may be linked to a toxin or to a detectable label, e.g. a radioactive label, non-radioactive isotopic label, fluorescent label, chemiluminescent label, paramagnetic label, enzyme label or calorimetric label. Those of ordinary skill in the art will know of these and other suitable labels which may be employed in accordance with the present invention. The binding of these labels to antibodies or fragments thereof can be accomplished using standard techniques commonly known to those of ordinary skill in the art.

It is also contemplated that pharmaceutical compositions may be prepared using the novel proteins of the present invention. In such a case, the pharmaceutical composition comprises the novel active composition(s) of the present invention and a pharmaceutically acceptable carrier. A person having ordinary skill in this art would readily be able to determine, without undue experimentation, the appropriate dosages and routes of administration of the active component of the present invention.

The phrase “pharmaceutically acceptable” refers to molecular entities and compositions that do not produce an allergic or similar untoward reaction when administered to a subject. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared. The preparation can also be emulsified.

A protein may be formulated into a composition in a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 mL of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

In one embodiment of the present invention, there are provided DNA sequences encoding a 30-kDa immunoreactive protein of Ehrlichia canis. Preferably, the protein has an amino acid sequence selected from the group consisting of SEQ ID No. 2, 4, 6, 40, 42, 44, 46, and the gene has a nucleic acid sequence selected from the group consisting of SEQ ID No. 1, 3, 5, 39, 41, 43, 45 and is a member of a polymorphic multiple gene family. More preferably, the protein has an N-terminal signal sequence which is cleaved after post-translational process resulting in the production of a mature 28-kDa protein. Still preferably, the DNAs encoding 28-kDa proteins are contained in a single multigene locus, which has the size of 10,677 bp and encodes nine homologous 28-kDa proteins of Ehrlichia canis.

In another embodiment of the present invention, there is provided an expression vector comprising a gene encoding a 28-kDa immunoreactive protein of Ehrlichia canis and capable of expressing the gene when the vector is introduced into a cell.

In still another embodiment of the present invention, there is provided a recombinant protein comprising an amino acid sequence selected from the group consisting of SEQ ID No. 2, 4, 6, 40, 42, 44, 46. Preferably, the amino acid sequence is encoded by a nucleic acid sequence selected from the group consisting of SEQ ID No. 1, 3, 5, 39, 41, 43, 45. More preferably, the recombinant protein comprises four variable regions which are surface exposed, hydrophilic and antigenic. Still preferably, the recombinant protein is an antigen.

In yet another embodiment of the present invention, there is provided a method of producing the recombinant protein, comprising the steps of obtaining a vector that comprises an expression region comprising a sequence encoding the amino acid sequence selected from the group consisting of SEQ ID No. 2, 4, 6, 40, 42, 44, 46 operatively linked to a promoter; transfecting the vector into a cell; and culturing the cell under conditions effective for expression of the expression region.

The invention may also be described in certain embodiments as a method of inhibiting Ehrlichia canis infection in a subject comprising the steps of: identifying a subject suspected of being exposed to or infected with Ehrlichia canis; and administering a composition comprising a 28-kDa antigen of Ehrlichia canis in an amount effective to inhibit an Ehrlichia canis infection. The inhibition may occur through any means such as, i.e. the stimulation of the subject's humoral or cellular immune responses, or by other means such as inhibiting the normal function of the 28-kDa antigen, or even competing with the antigen for interaction with some agent in the subject's body.

The following examples are given for the purpose of illustrating various embodiments of the invention and are not meant to limit the present invention in any fashion.

### EXAMPLE 1

Sequencing Unknown 5′ and 3′ Regions of the ECa28-1 (p28-7) Gene Ehrlichiae and Purification

Ehrlichia canis (Florida strain and isolates Demon, DJ, Jake, and Fuzzy) were provided by Dr. Edward Breitschwerdt, (College of Veterinary Medicine, North Carolina State University, Raleigh, N.C.). E. canis (Louisiana strain) was provided by Dr. Richard E. Corstvet (School of Veterinary Medicine, Louisiana State University, Baton Rouge, La.) and E. canis (Oklahoma strain) was provided by Dr. Jacqueline Dawson (Centers for Disease Control and Prevention, Atlanta, Ga.). Propagation of ehrlichiae was performed in DH82 cells with DMEM supplemented with 10% bovine calf serum and 2 mM L-glutamine at 37° C. The intracellular growth in DH82 cells was monitored by presence of E. canis morulae using general cytologic staining methods. Cells were harvested when 100% of the cells were infected with ehrlichiae and were then pelleted in a centrifuge at 17,000×g for 20 min. Cell pellets were disrupted with a Braun-Sonic 2000 sonicator twice at 40W for 30 sec on ice. Ehrlichiae were purified as described previously (Weiss et al., 1975). The lysate was loaded onto discontinuous gradients of 42%-36%-30% renografin, and centrifuged at 80,000×g for 1 hr. Heavy and light bands containing ehrlichiae were collected and washed with sucrose-phosphate-glutamate buffer (SPG, 218 mM sucrose, 3.8 mM KH₂PO₄, 7.2 mM K₂HPO₄, 4.9 mM glutamate, pH 7.0) and pelleted by centrifugation.

Nucleic Acid Preparation

Ehrlichia canis genomic DNA was prepared by resuspending the renografin-purified ehrlichiae in 600 μl of 10 mM Tris-HCl buffer (pH 7.5) with 1% sodium dodecyl sulfate (SDS, w/v) and 100 ng/ml of proteinase K as described previously (McBride et al., 1996). This mixture was incubated for 1 hr at 56° C., and the nucleic acids were extracted twice with a mixture of phenol/chloroform/isoamyl alcohol (24:24:1). DNA was pelleted by absolute ethanol precipitation, washed once with 70% ethanol, dried and resuspended in 10 mM Tris (pH 7.5). Plasmid DNA was purified by using High Pure Plasmid Isolation Kit (Boehringer Mannheim, Indianapolis, Ind.), and PCR products were purified using a QIAquick PCR Purification Kit (Qiagen, Santa Clarita, Calif.).

Cloning of ECa28-1 (p28-7) Gene

The full length sequence of p28-7 gene was determined using a Universal GenomeWalker Kit (CLONTECH, Palo Alto, Calif.) according to the protocol supplied by the manufacturer. Genomic E. canis (Jake isolate) DNA was digested completely with five restriction enzymes (DraI, EcoRV, PνuII, ScaI, StuI) which produce blunt-ended DNA. An adapter (AP1) supplied in the kit was ligated to each end of E. canis DNA. The genomic libraries were used as templates to find the unknown DNA sequence of the p28-7 gene by PCR using a primer complementary to a known portion of the p28-7 sequence and a primer specific for the adapter AP1. Primers specific for p28-7 used for genome walking were designed from the known DNA sequence derived from PCR amplification of p28-7 with primers 793 (SEQ ID NO. 16) and 1330 (SEQ ID NO. 17). Primers 394 (5′-GCATTTCCACAGGATCATAGGTAA-3′; nucleotides 687-710, SEQ ID NO. 21) and 394C (5′-TTACCTATGATCCTGT GGAAATGC-3; nucleotides 710-687, SEQ ID NO. 22) were used in conjunction with supplied primer AP1 to amplify the unknown 5′ and 3′ regions of the p28-7 gene by PCR. A PCR product corresponding to the 5′ region of the p28-7 gene amplified with primers 394C and AP1 (2000-bp) was sequenced unidirectionally with primer 793C (5′-GAGTA ACCAACAGCTCCTGC-3′, SEQ ID No. 23). A PCR product corresponding to the 3′ region of the p28-7 gene amplified with primers 394 and AP1 (580-bp) was sequenced bidirectionally with the same primers. Noncoding regions on the 5′ and 3′ regions adjacent to the open reading frame were sequenced, and primers EC28OM-F (5′-TCTACTTTGCACTTCC ACTATTGT-3′, SEQ ID NO. 24) and EC28OM-R (5′-ATTCTTTTGCCACTATTT TTCTTT-3′, SEQ ID NO. 25) complementary to these regions were designed in order to amplify the entire p28-7 gene.

DNA Sequencing

DNA was sequenced with an ABI Prism 377 DNA Sequencer (Perkin-Elmer Applied Biosystems, Foster City, Calif.). The entire p28-7 genes of seven E. canis isolates (four from North Carolina, and one each from Oklahoma, Florida, and Louisiana) were amplified by PCR with primers EC28OM-F (SEQ ID No. 24) and EC28OM-R (SEQ ID No. 25) with a thermal cycling profile of 95° C. for 5 minutes, and 30 cycles of 95° C. for 30 seconds, 62° C. for 1 minutes, and 72° C. for 2 minutes and a 72° C. extension for 10 minutes. The resulting PCR products were bidirectionally sequenced with the same primers.

### EXAMPLE 2

PCR Amplification, Cloning, Sequencing and Expression of E. canis ECa28-1 (p28-7) Gene

Expression Vectors

The entire E. canis p28-7 gene was PCR-amplified with primers-EC28OM-F and EC28OM-R and cloned into pCR2.1-TOPO TA cloning vector to obtain the desired set of restriction enzyme cleavage sites (Invitrogen, Carlsbad, Calif.). The insert was excised from pCR2.1-TOPO with BstX 1 and ligated into pcDNA 3.1 eukaryotic expression vector (Invitrogen, Carlsbad, Calif.) designated pcDNA3.1/EC28 for subsequent studies. The pcDNA3.1/EC28 plasmid was amplified, and the gene was excised with a KpnI-XbaI double digestion and directionally ligated into pThioHis prokaryotic expression vector (Invitrogen, Carlsbad, Calif.). The clone (designated pThioHis/EC28) produced a recombinant thioredoxin fusion protein in Escherichia coli BL21. The recombinant fusion protein was crudely purified in the insoluble phase by centrifugation. The control thioredoxin fusion protein was purified from soluble cell lysates under native conditions using nickel-NTA spin columns (Qiagen, Santa Clarita, Calif.).

Western Blot Analysis

Recombinant E. canis p28-7 fusion protein was subjected to SDS-polyacrylamide gel electrophoresis (SDS-PAGE) on 4-15% Tris-HCl gradient gels (Bio-Rad, Hercules, Calif.) and transferred to pure nitrocellulose (Schleicher & Schuell, Keene, N.H.) using a semi-dry transfer cell (Bio-Rad, Hercules, Calif.). The membrane was incubated with convalescent phase antisera from an E. canis-infected dog diluted 1:5000 for 1 hour, washed, and then incubated with an anti-canine IgG (H & L) alkaline phosphatase-conjugated affinity-purified secondary antibody at 1:1000 for 1 hour (Kirkegaard & Perry Laboratories, Gaithersburg, Md.). Bound antibody was visualized with 5-bromo-4-chloro-3-indolyl phosphate/nitroblue tetrazolium (BCIP/NBT) substrate (Kirkegaard & Perry Laboratories, Gaithersburg, Md.).

Southern Blot Analysis

To determine if multiple genes homologous to the p28-7 gene were present in the E. canis genome, a genomic Southern blot analysis was performed using a standard procedure (Sambrook et al. 1989). E. canis genomic DNA digested completely with each of the restriction enzymes BanII, EcoRV, HaeII, KpnI and SpeI, which do not cut within the p28-7 gene, and AseI which digests p28-7 at nucleotides 34, 43 and 656. The probe was produced by PCR amplification with primers EC28OM-F and EC28OM-R and digoxigenin (DIG)-labeled deoxynucleotide triphosphates (dNTPs) (Boehringer Mannheim, Indianapolis, Ind.) and digested with AseI. The digested probe (566-bp) was separated by agarose gel electrophoresis, gel-purified and then used for hybridization. The completely digested genomic E. canis DNA was electrophoresed and transferred to a nylon membrane (Boehringer Mannheim, Indianapolis, Ind.) and hybridized at 40° C. for 16 hr with the p28-7 gene DIG-labeled probe in DIG Easy Hyb buffer according to the manufacturer's protocol (Boehringer Mannheim, Indianapolis, Ind.). Bound probe was detected with a anti-DIG alkaline phosphatase-conjugated antibody and a luminescent substrate (Boehringer Mannheim, Indianapolis, Ind.) and exposed to BioMax scientific imaging film (Eastman Kodak, Rochester, N.Y.).

Sequence Analysis and Comparasion

E. chaffeensis p28 and C. ruminantium map-1 DNA sequences were obtained from the National Center of Biotechnology Information (NCBI). Nucleotide and deduced amino acid sequences, and protein and phylogenetic analyses were performed with LASERGENE software (DNASTAR, Inc., Madison, Wis.). Analysis of post-translational processing was performed by the method of McGeoch and von Heijne for signal sequence recognition using the PSORT program (McGeoch, 1985; von Heijne, 1986)

Sequence analysis of p28-7 from seven different strains of E. canis was performed with primers designed to amplify the entire gene. Analysis revealed the sequence of this gene was conserved among the isolates from North Carolina (four), Louisiana, Florida and Oklahoma.

Results

Alignment of nucleic acid sequences from E. chaffeensis p28 and Cowdria ruminantium map-1 using the Jotun-Hein aligorithm produced a consensus sequence with regions of high homology (>90%). These homologous regions (nucleotides 313-332 and 823-843 of C. ruminantium map-1; 307-326 and 814-834 of E. chaffeensis p28) were targeted as primer annealing sites for PCR amplification. PCR amplification of the E. canis p28-7 gene was accomplished with primers 793 (5-GCAGGAGCTGTTGGTTACTC-3′) (SEQ ID NO. 16) and 1330 (5′-CCTTCCTCCAAGTTCTATGCC-3′) (SEQ ID NO. 17), resulting in a 518-bp PCR product. E. canis DNA was amplified with primers 793 and 1330 with a thermal cycling profile of 95° C. for 2 min, and 30 cycles of 95° C. for 30 sec, 62° C. for 1 min, 72° C. for 2 min followed by a 72° C. extension for 10 min and 4° C. hold. The nucleic acid sequence of the E. canis PCR product was obtained by sequencing the product directly with primers 793 and 1330.

Analysis of the sequence revealed an open reading frame encoding a protein of 170 amino acids, and alignment of the 518-bp sequence obtained from PCR amplification of E. canis with the DNA sequence of E. chaffeensis p28 gene revealed a similarity greater than 70%, indicating that the genes were homologous.

Adapter PCR with primers 394 and 793C was performed to determine the 5′ and 3′ segments of the sequence of the entire gene. Primer 394 produced four PCR products (3-kb, 2-kb, 1-kb, and 0.8-kb), and the 0.8-bp product was sequenced bidirectionally using primers 394 and AP1. The deduced sequence overlapped with the 3′ end of the 518-bp product, extending the open reading frame 12-bp to a termination codon. An additional 625-bp of non-coding sequence at the 3′ end of the p28-7 gene was also sequenced.

Primer 394C was used to amplify the 5′ end of the p28-7 gene with supplied primer AP1. Amplification with these primers resulted in three PCR products (3.3, 3-kb, and 2-kb). The 2-kb fragment was sequenced unidirectionally with primer 793C. The sequence provided the putative start codon of the p28-7 gene and completed the 834-bp open reading frame encoding a protein of 278 amino acids. An additional 144-bp of readable sequence in the 5′ noncoding region of the p28-7 gene was generated. Primers EC28OM-F and EC28OM-R were designed from complementary non-coding regions adjacent to the p28-7 gene.

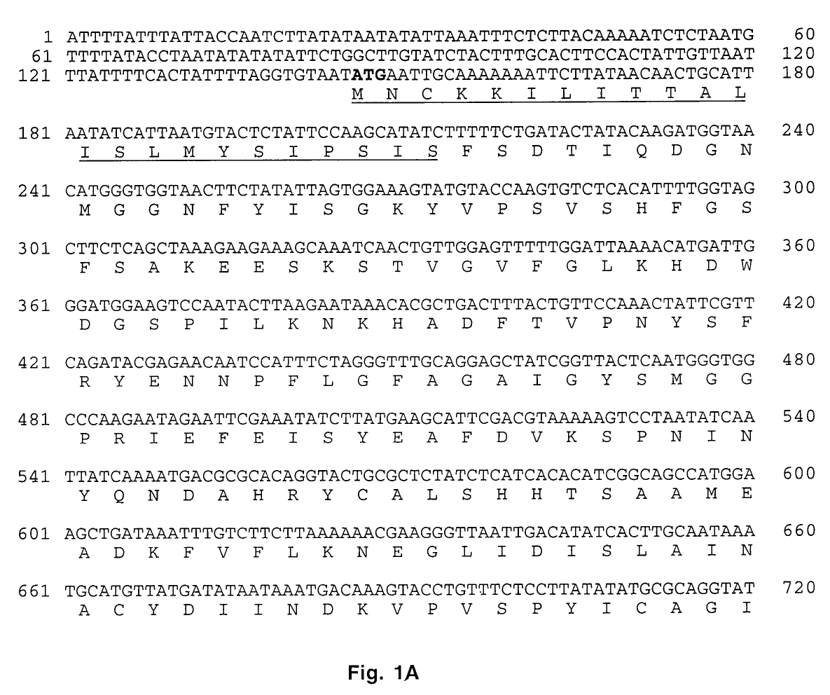

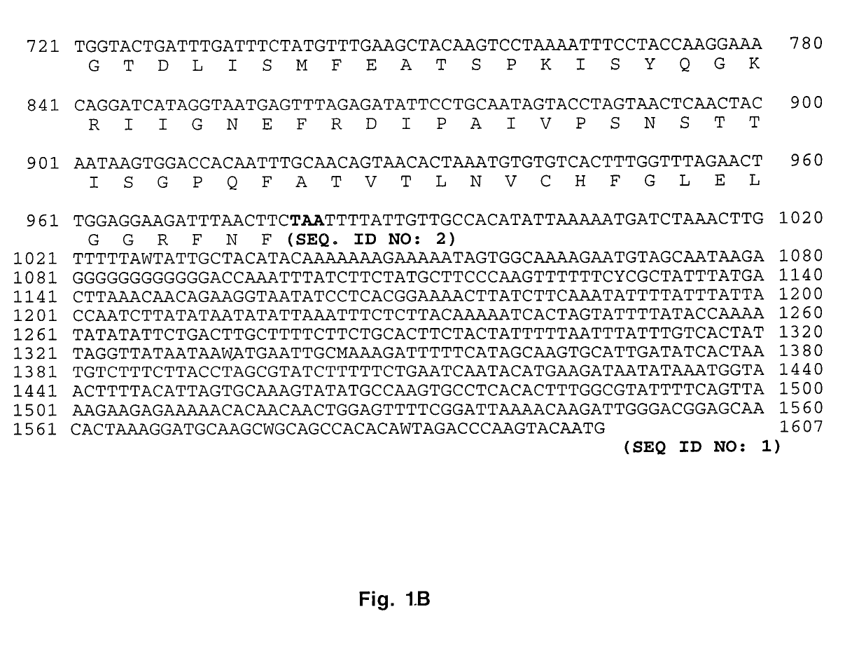

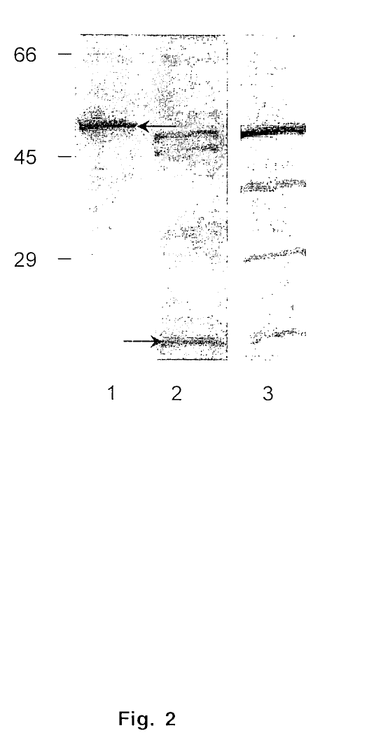

The PCR product amplified with these primers was sequenced directly with the same primers. The complete DNA sequence for the E. canis p28-7 gene (SEQ ID NO. 1) is shown in FIG. 1. The p28-7 PCR fragment amplified with these primers contained the entire open reading frame and 17 additional amino acids from the 5′ non-coding primer region. The gene was directionally subcloned into pThioHis expression vector, and E. coli (BL21) were transformed with this construct. The expressed p28-7-thioredoxin fusion protein was insoluble. The expressed protein had an additional 114 amino acids associated with the thioredoxin, 5 amino acids for the enterokinase recognition site, and 32 amino acids from the multiple cloning site and 5′ non-coding primer region at the N-terminus. Convalescent-phase antiserum from an E. canis infected dog recognized the expressed recombinant fusion protein, but did not react with the thioredoxin control (FIG. 2).

### EXAMPLE 3

Sequence Homology of E. canis p228-7 Gene

The nucleic acid sequence of E. canis p28-7 (834-bp) and the E. chaffeensis omp-1 family of genes including signal sequences (p28-7, omp-1A, B, C, D, E, and F) were aligned using the Clustal method to examine homology between these genes (alignment not shown). Nucleic acid homology was equally conserved (68.9%) between E. canis p28-7, E. chaffeensis p28 and omp-1F. Other putative outer membrane protein genes in the E. chaffeensis omp-1 family, omp-1D (68.2%), omp-1E (66.7%), omp-1C (64.1%), Cowdria ruminantium map-1 (61.8%), E. canis 28-kDa protein 1 gene (60%) and 28-kDa protein 2 gene (partial) (59.5%) were also homologous to p28-7. E. chaffeensis omp-1B had the least nucleic acid homology (45.1%) with E. canis p28-7.

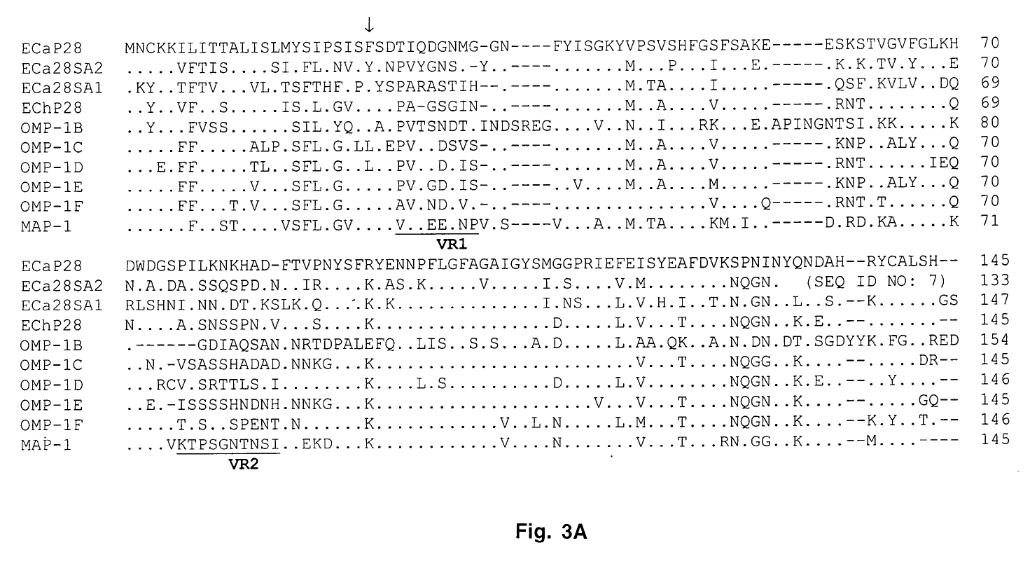

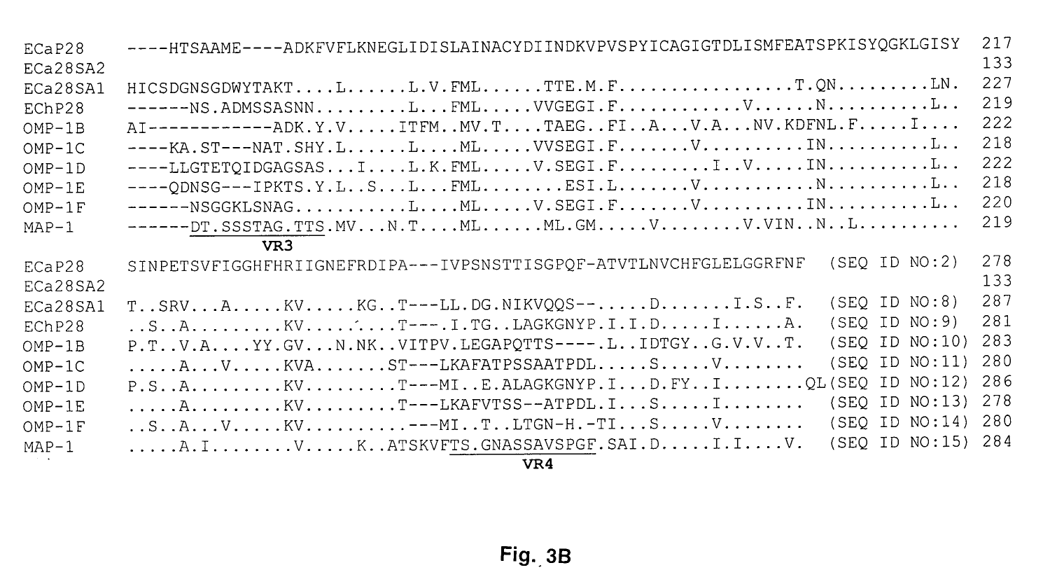

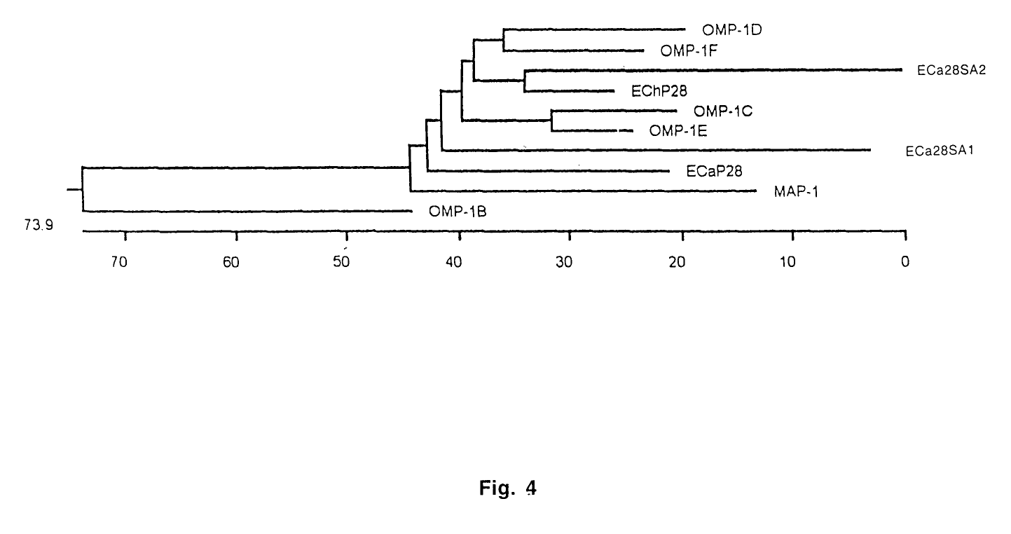

Alignment of the predicted amino acid sequences of E. canis P28-7 (SEQ ID NO. 2) and E. chaffeensis P28 revealed amino acid substitutions resulting in four variable regions (VR). Substitutions or deletions in the amino acid sequence and the locations of variable regions of E. canis P28-7 and the E. chaffeensis OMP-1 family were identified (FIG. 3). Amino acid comparison including the signal peptide revealed that E. canis P28-7 shared the most homology with OMP-1F (68%) of the E. chaffeensis OMP-1 family, followed by E. chaffeensis P28 (65.5%), OMP-1E (65.1%), OMP-1D (62.9%), OMP-1C (62.9%), Cowdria ruminantium MAP-1 (59.4%), E. canis 28-kDa protein 1 (55.6%) and 28-kDa protein 2 (partial) (53.6%), and OMP-1B (43.2%). The phylogenetic relationships based on amino acid sequences show that E. canis P28-7 and C. ruminantium MAP-1, E. chaffeensis OMP-1 proteins, and E. canis 28-kDa proteins 1 and 2 (partial) are related (FIG. 4).

### EXAMPLE 4

Predicted Surface Probability and Immunoreactivity of E. canis P28-7

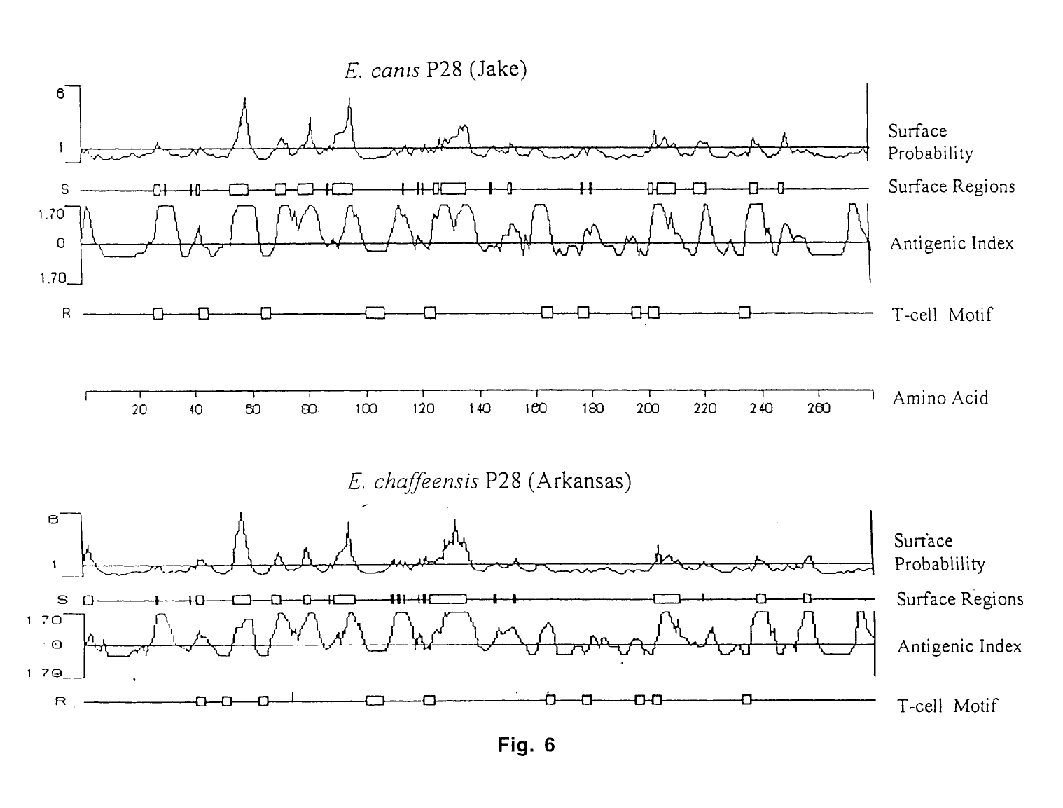

Analysis of E. canis P28-7 using hydropathy and hydrophilicity profiles predicted surface-exposed regions on P28-7 (FIG. 6). Eight major surface-exposed regions consisting of 3 to 9 amino acids were identified on E. canis P28-7 and were similar to the profile of surface-exposed regions on E. chaffeensis P28 (FIG. 6). Five of the larger surface-exposed regions on E. canis P28-7 were located in the N-terminal region of the protein. Surface-exposed hydrophilic regions were found in all four of the variable regions of E. canis P28-7. Ten T-cell motifs were predicted in the P28-7 using the Rothbard-Taylor aligorithm (Rothbard and Taylor, 1988), and high antigenicity of the E. canis P28-7 was predicted by the Jameson-Wolf antigenicity aligorithm (FIG. 6) (Jameson and Wolf, 1988). Similarities in antigenicity and T-cell motifs were observed between E. canis P28-7 and E. chaffeensis P28.

### EXAMPLE 5

Detection of Homologous Genomic Copies of E. canis p28-7 Gene

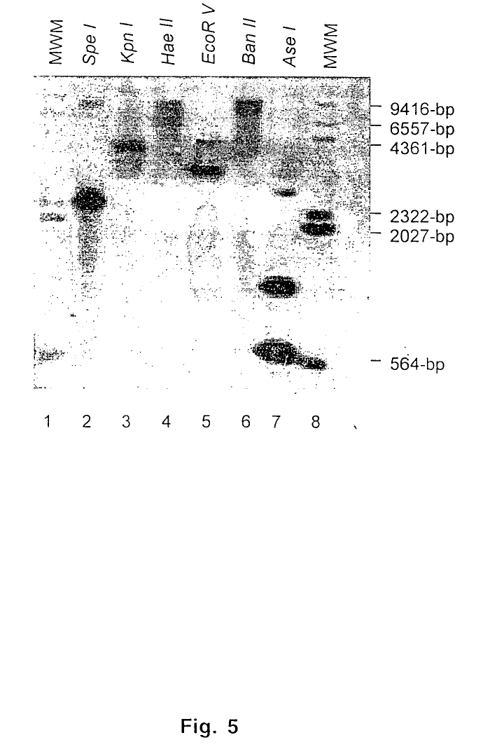

Genomic Southern blot analysis of E. canis DNA completely digested independently with restriction enzymes BanII, EcoRV, HaeII, KpnI, SpeI, which do not have restriction endonuclease sites in the p28-7 gene, and AseI, which has internal restriction endonuclease sites at nucleotides 34, 43 and 656, revealed the presence of at least three homologous p28-7 gene copies (FIG. 5). Although E. canis p28-7 has internal Ase I internal restriction sites, the DIG-labeled probe used in the hybridization experiment targeted a region of the gene within a single DNA fragment generated by the AseI digestion of the gene. Digestion with AseI produced 3 bands (approximately 566-bp, 850-bp, and 3-kb) that hybridized with the p28-7 DNA probe indicating the presence of multiple genes homologous to p28-7 in the genome. Digestion with EcoRV and SpeI produced two bands that hybridized with the p28-7 gene probe.

### EXAMPLE 6

PCR Amplification of E. canis ECa28SA2 (p28-5). ECa28SA3 (p28-6) Genes and Identification of the Multiple Gene Locus

In order to specifically amplify possible unknown genes downstream of ECa28SA2 (p28-5), primer 46f specific for p28-5 (5′-ATATACTTCCTACCTAATGTCTCA-3′, SEQ ID No. 18), and primer 1330 (SEQ ID No. 17) which targets a conserved region on the 3′ end of p28-7 gene were used for amplification. The amplified product was gel purified and cloned into a TA cloning vector (Invitrogen, Santa Clarita, Calif.). The clone was sequenced bidirectionally with primers: M13 reverse from the vector, 46f, ECa28SA2 (5′-AGTGCAGAGTCTTCGGTTTC-3′, SEQ ID No. 19), ECa5.3 (5′-GTTACTTGCGGAGGACAT-3′, SEQ ID No. 20). DNA was amplified with a thermal cycling profile of 95° C. for 2 min, and 30 cycles of 95° C. for 30 sec, 48° C. for 1 min, 72° C. for 1 min followed by a 72° C. extension for 10 min and 4° C. hold.

A 2-kb PCR product was amplified with these primers that contained 2 open reading frames. The first open reading frame contained the known region of the p28-5 gene and a previously unsequenced 3′ portion of the gene. Downstream from p28-5 an additional non identical, but homologous 28-kDa protein gene was found, and designated ECa28SA3 (p28-6).

Specific primers designated ECaSA3-2 (5′-CTAGGATTA GGTTATAGTATAAGTT-3′, SEQ ID No. 26) corresponding to regions within p28-6 and primer 793C (SEQ ID No. 23) which anneals to a region with p28-7 were used to amplify the intergenic region between gene p28-6 and p28-7. DNA was amplified with a thermal cycling profile of 95° C. for 2 min, and 30 cycles of 95° C. for 30 sec, 50° C. for 1 min, 72° C. for 1 min followed by a 72° C. extension for 10 min and 4° C. hold.

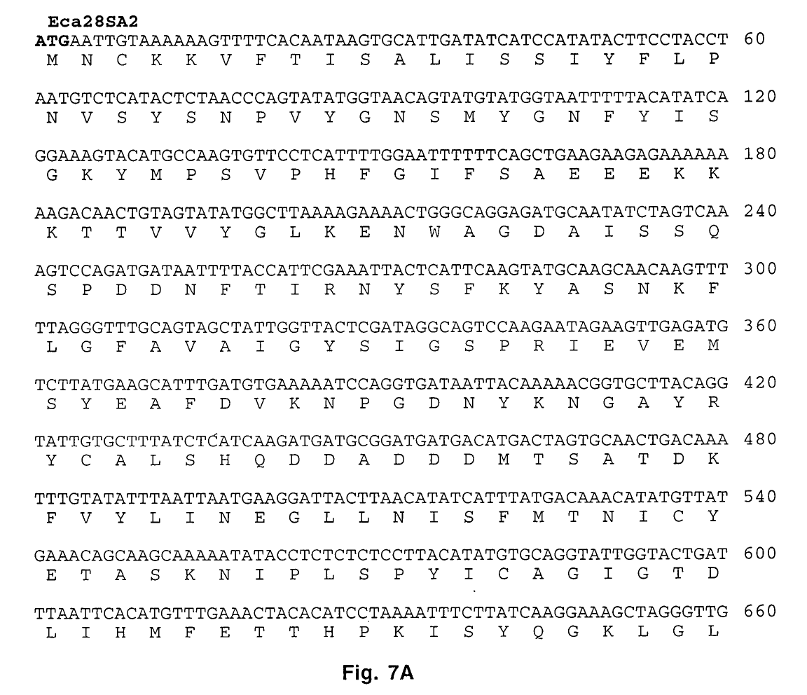

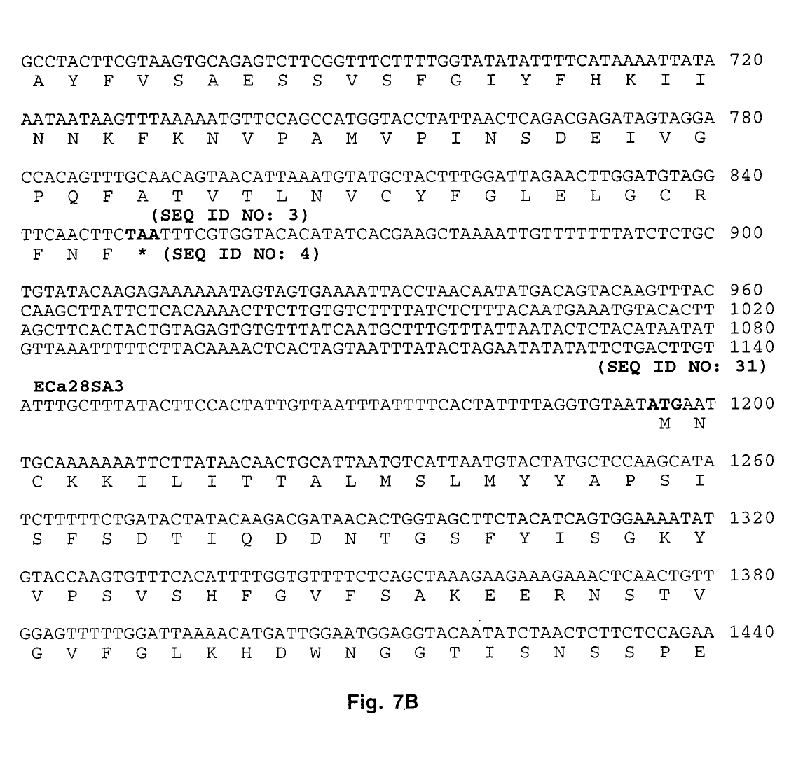

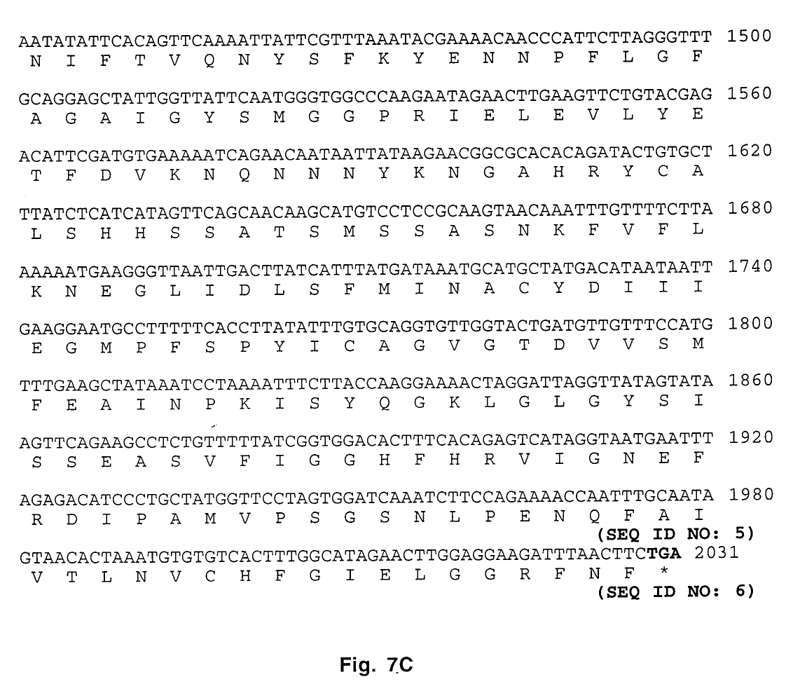

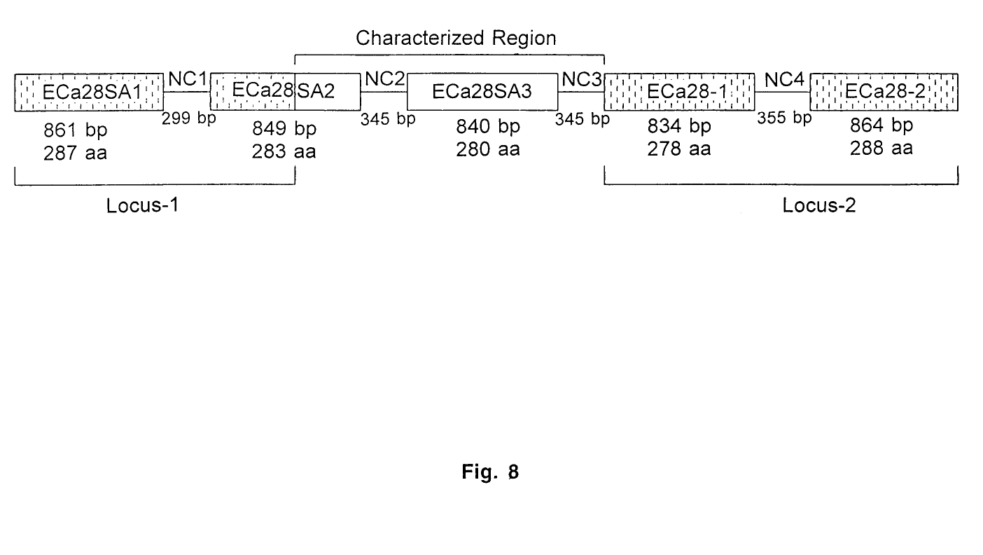

An 800-bp PCR product was amplified which contained the 3′ end of p28-6, the intergenic region between p28-6 and p28-7 (28NC3) and the 5′ end of p28-7, joining the previously separate loci (FIG. 8). The 849-bp open reading frame of p28-5 encodes a 283 amino acid protein, and p28-6 has an 840-bp open reading frame encoding a 280 amino acid protein. The intergenic noncoding region between p28-6 and p28-7 was 345-bp in length (FIGS. 7 and 8)

### EXAMPLE 7

Nucleic and Amino Acid Homology of E. canis p28-4, p28-5, p28-6, p28-7 and p28-8 Proteins

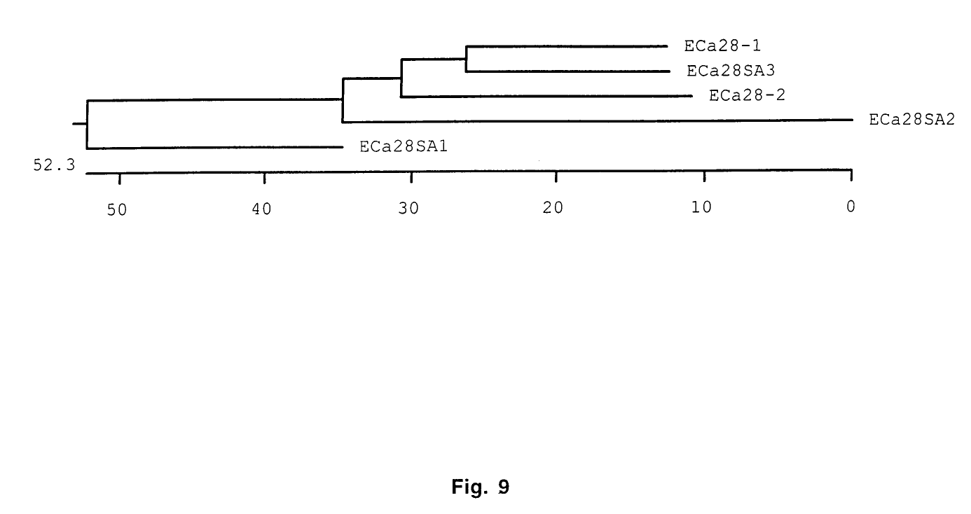

The nucleic and amino acid sequences of all five E. canis 28-kDa protein genes were aligned using the Clustal method to examine the homology between these genes. The nucleic acid homology ranged from 58 to 75% and a similar amino acid homology of ranging from 67 to 72% was observed between the E. canis 28-kDa protein gene members (FIG. 9).

Transcriptional Promoter Regions

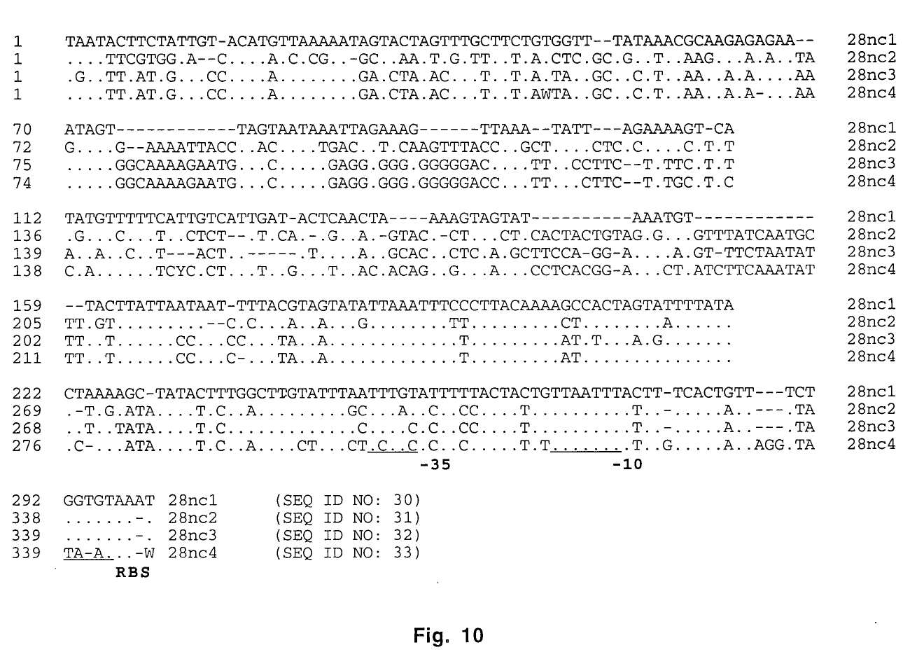

The intergenic regions between the 28-kDa protein genes were analyzed for promoter sequences by comparison with consensus Escherichia coli promoter regions and a promoter from E. chaffeensis (Yu et al., 1997; McClure, 1985). Putative promoter sequences including RBS, −10 and −35 regions were identified in 4 intergenic sequences corresponding to genes p28-5, p28-6, p28-7, and p28-8 (ECa28-2) (FIG. 10). The upstream noncoding region of p28-4 (ECa28SA1) is not known and was not analyzed.

N-Terminal Signal Sequence

The amino acid sequence analysis revealed that entire E. canis p28-7 has a deduced molecular mass of 30.5-kDa and the entire p28-6 has a deduced molecular mass of 30.7-kDa. Both proteins have a predicted N-terminal signal peptide of 23 amino acids (MNCKKILITTALMSLMYYAPSIS, SEQ ID No. 27), which is similar to that predicted for E. chaffeensis P28 (MNYKKILITSALISLISSLPGV SFS, SEQ ID NO. 28), and the OMP-1 protein family (Yu et al., 1999a; Ohashi et al., 1998b).

A preferred cleavage site for signal peptidases (SIS; Ser-X-Ser) (Oliver, 1985) is found at amino acids 21, 22, and 23 of p28-7. An additional putative cleavage site at amino acid position 25 (MNCKKILITTALISLMYSIPSISSFS, SEQ ID NO. 29) identical to the predicted cleavage site of E. chaffeensis P28 (SFS) was also present, and would result in a mature p28-7 with a predicted molecular mass of 27.7-kDa. Signal cleavage site of the previously reported partial sequence of p28-5 is predicted at amino acid 30. However, signal sequence analysis predicted that p28-4 had an uncleavable signal sequence.

### SUMMARY

Proteins of similar molecular mass have been identified and cloned from multiple rickettsial agents including E. canis, E. chaffeensis, and C. ruminantium (Reddy et al., 1998; Jongejan et al., 1993; Ohashi et al., 1998). A single locus in Ehrlichia chaffeensis with 6 homologous p28 genes, and 2 loci in E. canis, each containing some homologous 28-kDa protein genes have been previously described.

The present invention demonstrated the cloning, expression and characterization of genes encoding mature 28-kDa proteins of E. canis that are homologous to the omp-1 multiple gene family of E. chaffeensis and the C. ruminantium map-1 gene. Two new 28-kDa protein genes were identidfied, p28-7 and p28-6. Another E. canis 28-kDa protein gene, p28-5, partially sequenced previously (Reddy et al., 1998), was sequenced completely in the present invention. Also disclosed is the identification and characterization of a single locus in E. canis containing five E. canis 28-kDa protein genes (p28-4, p28-5, p28-6, p28-7 and p28-8).

The E. canis 28-kDa proteins are homologous to E. chaffeensis OMP-1 family and the MAP-1 protein of C. rumanintium. The most homologous E. canis 28-kDa proteins (p28-6, p28-7 and p28-8) are sequentially arranged in the locus. Homology of these proteins ranged from 67.5% to 72.3%. Divergence among these 28-kDa proteins was 27.3% to 38.6%. E. canis 28-kDa proteins p28-4 and p28-5 were the least homologous with homology ranging from 50.9% to 59.4% and divergence of 53.3 to 69.9%. Differences between the genes lies primarily in the four hypervariable regions and suggests that these regions are surface exposed and subject to selective pressure by the immune system. Conservation of p28-7 among seven E. canis isolates has been reported (McBride et al., 1999), suggesting that E. canis may be clonal in North America. Conversely, significant diversity of p28 among E. chaffeensis isolates has been reported (Yu et al., 1999a).

All of the E. canis 28-kDa proteins appear to be post translationally processed from a 30-kD protein to a mature 28-kD protein. Recently, a signal sequence was identified on E. chaffeensis P28 (Yu et al., 1999a), and N-terminal amino acid sequencing has verified that the protein is post-translationally processed resulting in cleavage of the signal sequence to produce a mature protein (Ohashi et al., 1998). The leader sequences of OMP-1F and OMP-1E have also been proposed as leader signal peptides (Ohashi et al., 1998). Signal sequences identified on E. chaffeensis OMP-1F, OMP-1E and P28 are homologous to the leader sequence of E. canis 28-kDa protein. Promoter sequences for the p28 genes have not been determined experimentally, but putative promoter regions were identified by comparison with consensus sequences of the RBS, −10 and −35 promoter regions of E. coli and other ehrlichiae (Yu et al., 1997; McClure, 1985). Such promoter sequences would allow each gene to potentially be transcribed and translated, suggesting that these genes may be differentially expressed in the host. Persistence of infection in dogs may be related to differential expression of p28 genes resulting in antigenic changes in vivo, thus allowing the organism to evade the immune response.

The E. canis 28-kda protein genes were found to exhibit nucleic acid and amino acid sequence homology with the E. chaffeensis omp-1 gene family and C. ruminantium map-1 gene. Previous studies have identified a 30-kDa protein of E. canis that reacts with convalescent phase antisera against E. chaffeensis, but was believed to be antigenically distinct (Rikihisa et al., 1994). Findings based on comparison of amino acid substitutions in four variable regions of E. canis 28-kDa proteins support this possibility. Together these findings also suggest that the amino acids responsible for the antigenic differences between E. canis and E. chaffeensis P28 are located in these variable regions and are readily accessible to the immune system.

It was reported that immunoreactive peptides were located in the variable regions of the 28-kDa proteins of C. ruminantium, E. chaffeensis and E. canis (Reddy et al., 1998). Analysis of E. canis and E. chaffeensis P28 revealed that all of the variable regions have predicted surface-exposed amino acids. A study in dogs demonstrated lack of cross protection between E. canis and E. chaffeensis (Dawson and Ewing, 1992). This observation may be related to antigenic differences in the variable regions of P28 as well as in other immunologically important antigens of these ehrlichial species. Another study found that convalescent phase human antisera from E. chaffeensis-infected patients recognized 29/28-kDa protein(s) of E. chaffeensis and also reacted with homologous proteins of E. canis (Chen et al., 1997). Homologous and crossreactive epitopes on the E. canis 28-kDa protein and E. chaffeensis P28 appear to be recognized by the immune system.

E. canis 28-kDa proteins may be important immunoprotective antigens. Several reports have demonstrated that the 30-kDa antigen of E. canis exhibits strong immunoreactivity (Rikihisa et al., 1994; Rikihisa et al., 1992). Antibodies in convalescent phase antisera from humans and dogs have consistently reacted with proteins in this size range from E. chaffeensis and E. canis, suggesting that they may be important immunoprotective antigens (Rikihisa et al., 1994; Chen et al., 1994; Chen et al., 1997). In addition, antibodies to 30, 24 and 21-kDa proteins developed early in the immune response to E. canis (Rikihisa et al., 1994; Rikihisa et al., 1992), suggesting that these proteins may b e especially important in the immune responses in the acute stage of disease. Recently, a family of homologous genes encoding outer membrane proteins with molecular masses of 28-kDa have been identified in E. chaffeensis, and mice immunized with recombinant E. chaffeensis P28 appeared to have developed immunity against homologous challenge (Ohashi et al., 1998). The P28 of E. chaffeensis has been demonstrated to be present in the outer membrane, and immunoelectron microscopy has localized the P28 on the surface on the organism, and thus suggesting that it may serve as an adhesin (Ohashi et al., 1998). It is likely that the 28-kDa proteins of E. canis identified in this study have the same location and possibly serve a similar function.

Comparison of p28-7 from different strains of E. canis revealed that the gene is apparently completely conserved. Studies involving E. chaffeensis have demonstrated immunologic and molecular evidence of diversity. Patients infected with E. chaffeensis have variable immunoreactivity to the 29/28-kDa proteins, suggesting that there is antigenic diversity (Chen et al., 1997). Recently molecular evidence has been generated to support antigenic diversity in the p28 gene from E. chaffeensis (Yu et al., 1999a). A comparison of five E. chaffeensis isolates revealed that two isolates (Sapulpa and St. Vincent) were 100% identical, but three others (Arkansas, Jax, 91HE17) were divergent by as much as 13.4% at the amino acid level. The conservation of E. canis p28-7 suggests that E. canis strains found in the United States may be genetically identical, and thus E. canis 28-kDa protein is an attractive vaccine candidate for canine ehrlichiosis in the United States. Further analysis of E. canis isolates outside the United States may provide information regarding the origin and evolution of E. canis. Conservation of the 28-kDa protein makes it an important potential candidate for reliable serodiagnosis of canine ehrlichiosis.

The role of multiple homologous genes is not known at this point; however, persistence of E. canis infections in dogs could conceivably be related to antigenic variation due to variable expression of homologous 28-kDa protein genes, thus enabling E. canis to evade immune surveillance. Variation of msp-3 genes in A. marginale is partially responsible for variation in the MSP-3 protein, resulting in persistent infections (Alleman et al., 1997). Studies to examine 28-kDa protein gene expression by E. canis in acutely and chronically infected dogs would provide insight into the role of the 28-kDa protein gene family in persistence of infection.

### EXAMPLE 8

Identification of E. canis p228-1, p28-2, p28-3 and p28-9 Genes

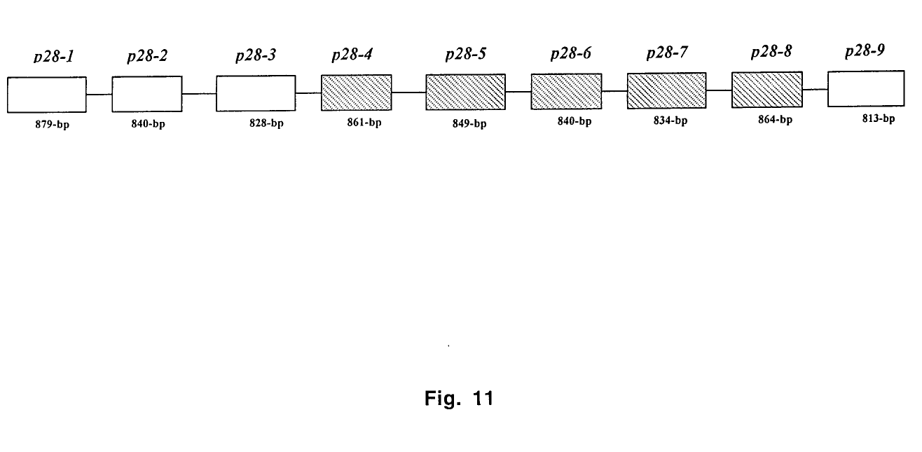

Unknown regions of DNA upstream and downstream of the five gene locus of tandemly arranged p28 genes described above were sequenced by designing gene specific primers for p28-1 (ECa28-75C) and p28-5 (ECa28-5-818f) to extend the p28 gene locus bidirectionally. Multiple gene walks were performed to obtain the unknown sequence as follows: 1.9-kp downstream of the 5 gene locus was amplified and sequenced using primers p28-5-818f (5′-TTA AAC ATA TGC CAC TTC GGA CTA-3′, SEQ ID No. 34), producing a 900-bp amplicon, and 1191 (5′-TAT GAT CGT GTA AAA TTG CTG TGA GTA T-3′, SEQ ID No. 35), producing a 1-kb amplicon. The 3.67-kbp of DNA upstream of the five gene locus was amplified and sequenced with primers ECa28-75C (5′-TAC TGG CAC GTG CTG GAC TA-3′, SEQ ID No. 36), producing a 1.6-kbp amplicon; ECa5′-1600 (5′-CAC CAA TAA ATG CAG AGA CTT C-3′, SEQ ID No. 37), producing a 1.6-kbp amplicon; and 3125 (5′-AAT CCA TCA TTT CTC ATT ACA GTG TG-3′, SEQ ID No. 38), producing a 800-bp amplicon. The locus of nine tandemly arranged genes consisting of the four new p28 genes, and the five p28 genes described above were designated p28-1 through p28-9 (FIG. 11).

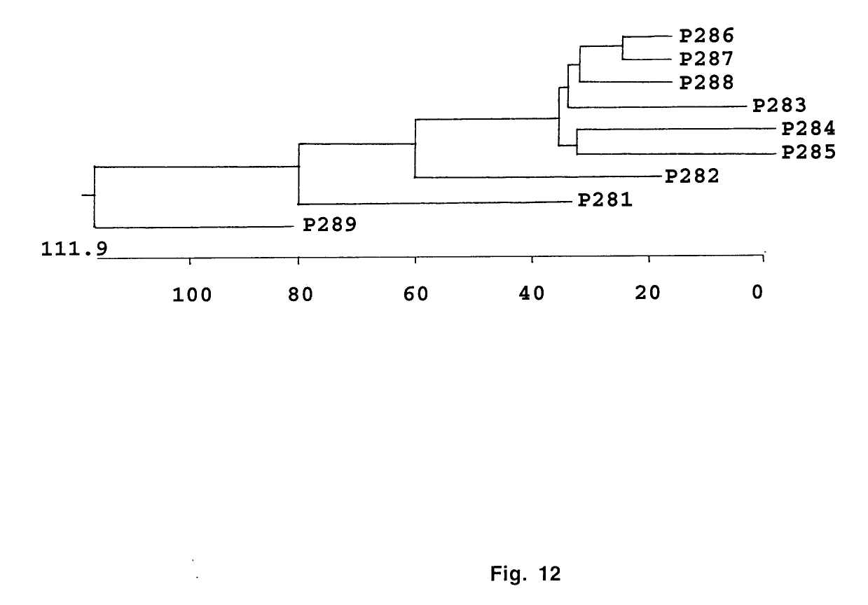

The nucleic acid and amino acid sequences of the E. canis p28 genes were aligned using the Clustal method to examine the homology between these genes. Homology of these proteins ranged from 67.5% to 75%, and divergence among these P28 proteins was 26.9% to 38%. E. canis P28 proteins P28-1, P28-2, and P28-9 were the least homologous with the other p28 genes ranging from 37% to 49% and divergence of 53 to 77%. The nucleic acid homology of the nine p28 genes ranged from 28 to 72%. The phylogenetic relationships based on the E. canis p28 amino acid sequences are shown in FIG. 12.

Nucleotide Sequence and Accession Numbers.

The GenBank accession numbers for the nucleic acid and amino acid sequences for the complete nine gene E. canis (Jake strain) p28 gene locus is AF082744. This accession number was originally assigned to p28-7, but has been updated with the sequence of the nine gene p28 locus, which includes p28-7. GenBank accession numbers for nucleic acid and amino acid sequences of p28-7 in other E. canis isolates described in this study are: Louisiana, AF082745; Oklahoma, AF082746; Demon, AF082747; DJ, AF082748; Fuzzy, AF082749; Florida, AF082750.

Multiple bands in the 28-kilodalton range have been observed by immunoblots of convalescent sera from E. canis infected dogs (Rikihisa et al., 1994), and expression of multiple p28 proteins could be an explanation for this observation. Southern blot studies suggest that other p28 genes, in addition to the five members of this locus, are present in the genome (McBride et al., 1999; Ohashi et al., 1998b).

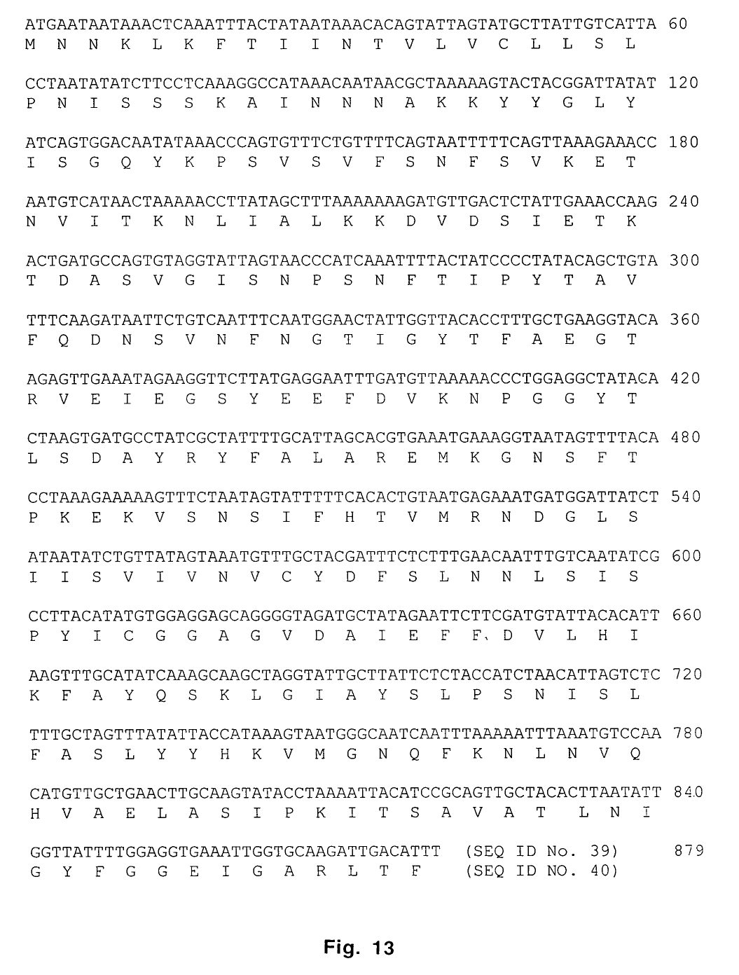

In this study a single gene locus containing nine tandemly arranged E. canis p28 genes encoding homologous, b u t nonidentical, p28 genes was identified. The nine gene locus included four new p28 genes (FIGS. 13-16) and five tandemly arranged p28 genes that were reported above. Eight of the p28 genes were located on one DNA strand, and one p28 gene was found on the complementary strand. The nucleic acid homology among the nine p28 gene members was 37 to 75%, and the amino acid homology ranged from 28 to 72%.

The P28s of E. canis were found to be as closely related to 28-kilodalton proteins of other species such as E. chaffeensis as they are to themselves (McBride et al., 2000). Differences among the proteins are found primarily in several major hypervariable regions and suggest that these regions are surface exposed and subject to selective pressure by the immune system (McBride et al., 2000).

Conservation of an E. canis p28 gene (p28-7) among seven geographically different isolates has been reported (McBride et al., 1999), suggesting that E. canis may be highly conserved in North America. Similarly, the 120-kDa glycoprotein of E. canis is also conserved among isolates in the United States (Yu et al., 1997). In contrast, both the 120-kDa and the 28-kDa protein genes of E. chaffeensis are divergent among isolates (Yu et al., 1999a; Chen et al., 1997). The diversity of the 28-kDa protein gene of E. chaffeensis appeared to result from point mutations in the hypervariable regions perhaps due to selective immune pressure (Yu et al., 1999a). These data suggest that E. canis may have been introduced into North America relatively recently, and this may account for the conservation that was observed among isolates. The conservation of p28 genes in E. canis isolates may provide an opportunity to develop vaccine and serodiagnostic antigens that are particularly effective for disease prevention and serodiagnosis. A mixture of the P28s may provide the most reliable serodiagnostic test, but serodiagnosis with a single P28 has been reported to be useful for immunodiagnosis (Ohashi et al., 1998b; McBride et al., 1999).

The following references were cited herein.

Alleman A. R., et al., (1997) Infect Immun 65: 156-163.

Anderson B. E., et al., (1991) J Clin Microbiol 29: 2838-2842.

Anderson B. E., et al., (1992) Int J Syst Bacteriol 42: 299-302.

Brouqui P., et al., (1992) J Clin Microbiol 30: 1062-1066.

Chen S. M., et al., (1997) Clin Diag Lab Immunol 4: 731-735.

Chen S. M., et al., (1994) Am J Trop Med Hyg 50: 52-58.

Dawson J. E., et al., (1992) Am J Vet Res 53: 1322-1327.

Dawson J. E., et al., (1991) J Infect Dis 163: 564-567.

Donatien, et al., (1935) Bull Soc Pathol Exot 28: 418-9.

Ewing, (1963) J Am Vet Med Assoc 143: 503-6.

Groves M. G., et al., (1975) Am J Vet Res 36: 937-940.

Harrus S., et al., (1998) J Clin Microbiol 36: 73-76.

Jameson B. A., et al., (1988) CABIOS 4: 181-186.

Jongejan F., et al., (1993) Rev Elev Med Vet Pays Trop 46: 145-152.

McBride J. W., et al., (1996) J Vet Diag Invest 8: 441-447.

McBride, et al., (1999) Clin Diagn Lab Immunol. 6: 392-399.

McBride, et al., (2000) Gene; In press

McClure, (1985) Ann Rev Biochem 54: 171-204.

McGeoch D. J. (1985) Virus Res 3: 271-286.

Nyindo M., et al., (1991) Am J Vet Res 52: 1225-1230.

Nyindo, et al., (1971) Am J Vet Res 32: 1651-58.

Ohashi, et al., (1998a) Infect Immun 66: 132-9.

Ohashi, et al., (1998b) J Clin Microb 36: 2671-80

Reddy, et al., (1998) Biochem Biophys Res Comm 247: 636-43.

Rikihisa, et al., (1994) J Clin Microbiol 32: 2107-12.

Rothbard J. B., et al., (1988) The EMBO J7: 93-100.

Sambrook J., et al., (1989) In Molecular Cloning: A Laboratory Manual. Cold Spring Harbor: Cold Spring Harbor Press.

Sulsona et al., (1999) Biochem. Biophys. Res. Commun. 257: 300-305.

Troy G. C., et al., (1990) Canine ehrlichiosis. In Infectious diseases of the dog and cat. Green C. E. (ed). Philidelphia: W. B. Sauders Co.

von Heijne, (1986) Nucl Acids Res 14: 4683-90.

Walker, et al., (1970) J Am Vet Med Assoc 157: 43-55.

Weiss E., et al., (1975) Appl Microbiol 30: 456-463.

Yu et al., (1993) J. Clin. Microbiol. 31: 3284-3288.

Yu, et al., (1997) Gene 184: 149-154.

Yu, et al., (1999a) J. Clin. Microbiol. 37: 1137-1143.

Yu et al., (2000) Gene 248: 59-68.

Any patents or publications mentioned in this specification are indicative of the levels of those skilled in the art to which the invention pertains. These patents and publications are herein incorporated by reference to the same extent as if each individual publication was individually indicated to be incorporated by reference.

One skilled in the art will readily appreciate that the present invention is well adapted to carry out the objects and obtain the ends and advantages mentioned, as well as those inherent therein. The present examples along with the methods, procedures, treatments, molecules, and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

What is claimed is:

1. A recombinant protein comprising an amino acid sequence selected from the group consisting of SEQ ID No. 2, 4, 6, 40, 42, 44 and 46.

2. The recombinant protein of claim 1, wherein said amino acid sequence is encoded by a nucleic acid segment comprising a sequence selected from the group consisting of SEQ ID No. 1, 3, 5, 39, 41, 43 and 45.
